(19) United States
(12) Patent Application Publication
(10) Pub. No.: US 2016/0222358 A1
(43) Pub. Date: Aug. 4, 2016

(54) PLANTS WITH INCREASED GROWTH OVER EXPRESSING A MITOCHONDRIAL GLYCINE DECARBOXYLASE COMPLEX SUBUNIT

(71) Applicant: BAYER CROPSCIENCE NV, B-Diegem (BE)

(72) Inventor: Hermann BAUWE, Rostock (DE)

(73) Assignee: Bayer CropScience NV, Diegem (BE)

(21) Appl. No.: 14/915,670

(22) PCT Filed: Sep. 4, 2013
(86) PCT No.: PCT/EP2013/068284

§ 371 Date: Mar. 1, 2016

(51) Int. Cl. C12N 9/06; C12N 15/82

(52) U.S. Cl. CPC C12N 9/0014; C12N 15/8269; C12N 15/8261; C12Y 104/04002

(57) ABSTRACT

The present invention relates to the field of plant molecular biology and concerns methods for enhancing photorespiration, photosynthesis, growth or yield in plants by modulating the expression of the glycine decarboxylase, also known as the glycine cleavage system. The present invention also provides recombinant constructs useful in the methods in the invention. In addition, the invention provides transgenic plants having an enhanced photorespiration, photosynthesis, growth or yield.

### FIELD OF THE INVENTION

The current invention relates to the field of molecular biology, specifically the field of agricultural biology. In particular, the invention relates to increased photosynthesis and/or photorespiration by modulating the activity of a subunit of the glycine cleavage system, (also known as glycine decarboxylase system), preferably by overexpression of the H-protein under control of a light-inducible promoter, such as a light-inducible promoter which is selectively expressed in green-tissue, leading to increased plant growth and yield.

### INCORPORATION OF SEQUENCE LISTING

The sequence listing that is contained in the file named BCS13-2014_ST25.txt, which is 8.87 kilobytes (measured in MS windows operating system), comprises sequences 1 to 5 and was created on Aug. 5, 2013, is filed herewith and incorporated herein by reference.

### BACKGROUND OF THE INVENTION

As a close partner of the Calvin-Benson (CB) cycle of photoautotrophic CO₂ fixation, the photorespiratory cycle is one of the major highways for the flow of carbon in the geo-biosphere. Briefly, this metabolic process starts when the key enzyme of the Calvin-Benson cycle, ribulose 1,5-bisphosphate (RuBP) carboxylase/oxygenase (Rubisco), covalently binds O₂ instead of CO₂ to RuBP [1]. Oxygenation of RuBP then produces one molecule each of 3-phosphoglycerate (3PGA) and 2-phosphoglycolate (2PG). In plants grown in normal air, the chance for binding CO₂ is only about twice as high as for O₂; that is, about every third to fourth molecule of RuBP becomes oxygenated [2]. Consequently, most land plants produce huge amounts of 2PG every day, which cannot directly re-enter the CB cycle and is also a potent inhibitor of enzymes of the CB cycle [3,4]. It is scavenged by the photorespiratory cycle, which combines two molecules of 2PG to one molecule of 3PGA, releasing one molecule of CO₂ [5-7]. Over decades, much effort has been spent to engineer Rubisco with less oxygenase activity [8], reduce photorespiratory CO₂ losses by increasing re-assimilation [9] or improve C3 photosynthesis by other means [10,11]. The most ambitious contemporary project in this context is directed towards engineering a CO₂-concentrating C4 rice variant [12].

Photorespiration is a universal and vital feature of all oxygenic autotrophs including cyanobacteria, green microalgae, and C4 plants [13-15]. Intriguingly, even small impairments of photorespiratory carbon flow, may they be caused by chemical inhibitors [16] or genetic approaches [17,18], reduce photosynthetic CO2 fixation. The mechanism of this feedback is not exactly known but could include inhibition of key enzymes of the CB cycle by photorespiratory metabolites such as 2PG [3,4], glyoxylate [19-21], and glycine [22].

Srinivasan and Oliver (Plant Physiol., 1992, 98, 1518-1519) described the cloning of a cDNA encoding the H-protein of the glycine decarboxylase multienzyme complex.

Kopriva and Bauwe (Mol. Gen. Genet., 1995, 249: 111-116) described that the H-protein of glycine decarboxylase is encoded by multigene families in Flaveria pringlei and Flaveria cronquistii.

Bauwe and Kolukisaoglu (Journal of Experimental Botany, 2003, 54, 1523-1535) reviewed genetic manipulation of glycine decarboxylation in plants, including the description of mutants induced by chemical mutagenesis, as well as antisense plants with reduced contents of glycine decarboxylase subunits and serine hydroxylmethyltransferase.

WO2010/046221, entitled “Plants with increased yield (NUE)” describes methods for producing a plant with increased yield as compared to a corresponding wild type plant whereby the method comprises at least the following step: increasing or generating in a plant or a part thereof one or more activities selected from the group consisting of 17.6 kDa class I heat shock protein, 26.5 kDa class I small heat shock protein, 26S protease subunit, 2-Cys peroxiredoxin, 3-dehydroquinate synthase, 5-keto-D-gluconate-5-reductase, asparagine synthetase A, aspartate 1-decarboxylase precursor, ATP-dependent RNA helicase, B0567-protein, B1088-protein, B1289-protein, B2940-protein, calnexin homolog, CDS5399-protein, chromatin structure-remodeling complex protein, D-amino acid dehydrogenase, D-arabinono-1,4-lactone oxidase, Delta 1-pyrroline-5-carboxylate reductase, glycine cleavage complex lipoylprotein, ketodeoxygluconokinase, lipoyl synthase, low-molecular-weight heat-shock protein, Microsomal cytochrome b reductase, mitochondrial ribosomal protein, mitotic check point protein, monodehydroascorbate reductase, paraquat-inducible protein B, phosphatase, phosphoglucosamine mutase, protein disaggregation chaperone, protein kinase, pyruvate decarboxylase, recA family protein, rhodanese-related sulfurtransferase, ribonuclease P protein component, ribosome modulation factor, sensory histidine kinase, serine hydroxymethyltransferase, SLL1280-protein, SLL1797-protein, small membrane lipoprotein, Small nucleolar ribonucleoprotein complex subunit, Sulfatase, transcription initiation factor subunit, tretraspanin, tRNA ligase, xyloglucan galactosyltransferase, YKL130C-protein, YLR443Wprotein, YML096W-protein, and zinc finger family protein-activity. The document specifically describes overexpression of glycine cleavage complex lipoylprotein from E. coli (SEQ ID Nos 289/290) and further mentions in the sequence listing the nucleotide sequences of Flaveria H-protein in SEQ ID Nos 613 and 614.

WO2011/060920 entitled “Process for the production of fine chemicals” describes a process of the production of a fine chemical in a non-human organism, like a microorganism, a plant cell, a plant, a plant tissue or in one or more parts thereof. The document further describes nucleic acid molecules, polypeptides, nucleic acid constructs, expression cassettes, vectors, antibodies, host cells, plant tissue, propagation material, harvested material, plants, microorganisms as well as agricultural compositions and their use. Flaveria pringlei GDC H-protein is mentioned among a long lists of sequences as SEQ ID Nos. 127410 and 117217.

WO2011/080674 entitled “Isolated polynucleotides and polypeptides and methods of using same for increasing plant yield, biomass, growth rate, vigor, oil content, abiotic stress tolerance of plants and nitrogen use efficiency” describes isolated polynucleotides encoding a polypeptide at least 80% homologous to the amino acid sequence selected from the group consisting of SEQ ID NOs: 799,488-798,800-813,4852-5453,5460,5461, 5484,5486-5550,5553, and 5558-8091; and isolated polynucleotide comprising nucleic acid sequences at least 80% identical to SEQ ID NO: 460, 1-459, 461-487, 814-1598, 1600-1603, 1605-1626, 1632-1642, 1645-4850 or 4851. Also provided are nucleic acid constructs comprising same, isolated polypeptides encoded thereby, transgenic cells and transgenic plants comprising same and methods of using same for increasing yield, biomass, growth rate, vigor, oil content, fiber yield, fiber quality, abiotic stress tolerance, and/or nitrogen use efficiency of a plant. Also provided are isolated polynucleotides comprising the nucleic acid sequence set forth by SEQ ID NO: 8096, wherein the isolated polynucleotide is capable of regulating expression of at least one polynucleotide sequence operably linked thereto. SEQ ID 7979 corresponds to the amino acid sequence of a putative GDC H-protein of Vitis vinifera.

US2013/0097737 entitled “Nucleic acid molecules and other molecules associated with plants and uses thereof for plant improvement” describes recombinant polynucleotides and recombinant polypeptides useful for improvement of plants are provided. The disclosed recombinant polynucleotides and recombinant polypeptides find use in production of transgenic plants to produce plants having improved properties. SEQ ID 59937 corresponds to an amino acid sequence of putative GDC H-protein of Gossypium hirsutum.

US2012/0096584 entitled “Nucleotide sequences and polypeptides encoded thereby useful for modifying plant characteristics” describes isolated nucleic acid molecules and their corresponding encoded polypeptides able to confer the trait of modulated low light sensitivity and modulated flowering time. The document also describes the use of these nucleic acid molecules and polypeptides in making transgenic plants, plant cells, plant materials or seeds of a plant having such modulated growth or phenotype characteristics that are altered with respect to wild type plants grown under similar conditions. SEQ ID 2634 corresponds to an amino acid sequence of a putative GDC H-protein of Glycine max.

Timm et al., (FEBS Letters, 586(2012) 3692-3697 (the disclosure of which corresponds to parts of this document) entitled “Glycine decarboxylase controls photosynthesis and plant growth” describes that photorespiration makes oxygenic photosynthesis possible by scavenging 2-phosphoglycolate. Hence, compromising photorespiration impairs photosynthesis. The authors examined whether facilitating photorespiratory carbon flow in turn accelerates photosynthesis and found that overexpression of the H-protein of glycine decarboxylase indeed considerably enhanced net-photosynthesis and growth of Arabidopsis thaliana. At the molecular level, lower glycine levels confirmed elevated GDC activity in vivo, and lower levels of the CO₂ acceptor ribulose 1,5-bisphosphate indicated higher drain from CO₂ fixation. Thus, the photorespiratory enzyme glycine decarboxylase appears as an important feed-back signaller that contributes to the control of the Calvin-Benson cycle and hence carbon flow through both photosynthesis and photorespiration.

### SUMMARY OF THE INVENTION

In one embodiment, the invention provides a plant comprising a recombinant gene, the recombinant gene comprising the following operably linked DNA regions: a light-inducible plant-expressible promoter, a DNA region encoding a subunit of the mitochondrial glycine decarboxylase complex, such as the H-protein (glycine cleavage complex lipoylprotein) or alternatively such as the P-protein, the T-protein or the L-protein, and optionally, a 3′ end region involved in transcription termination and polyadenylation, preferably a 3′ end region functional in plant cells.

In one embodiment the H-protein may be an H-protein derived from a plant such as a seed-bearing plant including Aegilops tauschii, Arabidopsis lyrata, Arabidopsis thaliana, Beta vulgaris, Brachypodium distachyon, Cicer arietinum, Cucumis sativus, Flaveria anomala, Flaveria bidentis, Flaveria brownii, Flaveria chlorifolia, Flaveria cronquistii, Flaveria floridana, Flaveria linearis, Flaveria palmeri, Flaveria pringlei, Flaveria pubescens, Flaveria trinervia, Glycine max, Hordeum vulgare subsp. vulgare, Lotus japonica, Medicago truncatula, Oryza sativa Indica Group, Oryza sativa Japonica Group, Pinus pinaster, Pisum abyssinicum, Pisum fulvum, Pisum sativum subsp. elatius, Pisum sativum subsp. transcaucasicum, Pisum sativum var. pumilio, Pisum sativum var. tibetanicum, Pisum sativum, Populus tremuloides, Populus trichocarpa, Ricinus communis, Sonneratia alba, Sorghum bicolor, Sorghum bicolor, Triticum aestivum, Triticum urartu, Vitis vinifera or Zea mays or the H-protein may be an H-protein derived from an algal species including Micromonas or Chlamydomonas. In another embodiment the H-protein comprises an amino acid sequence having at least 80% sequence identity with the amino acid sequence of SEQ ID NO. 1.

In one embodiment, the light-inducible promoter may be a promoter of an LS1 gene, a promoter of Rubisco small subunit gene, or a promoter of a chlorophyll a/b binding protein gene. In another embodiment, the light-inducible promoter may comprise the nucleotide sequence of SEQ ID 3 from nucleotide 1 to nucleotide 1571. In yet another embodiment, the recombinant gene comprises a ST-LS1 promoter from Solanum tuberosum operably linked to a H-protein encoding region from Flaveria pringlei.

The invention also provides a plant with increased photosynthesis and/or photorespiration wherein the level of active GDC H-protein in the mitochondria has been increased compared to a wild-type plant, such as by using a recombinant gene expressing the H-protein under control of a heterologous promoter The plant may be oilseed rape, cotton, rice, soybean, wheat, sugarcane or corn.

It is also an object of the invention to provide recombinant genes as herein described.

In an alternative embodiment, the invention provides a method for increasing photosynthesis and/or photorespiration in a cell of a plant, a plant, or part of a plant comprising the step of providing a recombinant gene to cells of the plant, the recombinant gene comprising the following operably linked DNA fragments
a. a plant-expressible promoter;
b. a DNA region encoding a subunit of the mitochondrial glycine decarboxylase complex such as the H-protein (glycine cleavage complex lipoylprotein) or alternatively such as the P-protein, the T-protein or the L-protein; and
c. optionally, a transcription termination and polyadenylation region.

In yet another embodiment, the invention provides a method for increasing yield and/or biomass of a plant comprising the step of providing the cells of the plant with a recombinant gene as herein described.

The invention also provides a method for producing a plant with increased biomass or yield comprising the step of providing the cells of the plant with a recombinant gene as herein described and optionally regenerating cells of the plant into a plant.

It is also an object of the invention to provide use of an mitochondrial protein GDC H encoding DNA fragment to increase the photosynthesis and/or photorespiration in a plant or use of an mitochondrial protein H encoding DNA fragment to increase the yield and/or biomass in a plant.

The invention also provides as alternative embodiment a seed of a plant comprising a recombinant gene as herein described.

### BRIEF DESCRIPTION OF THE DRAWINGS

FIG. 1: Schematic representation of the overexpression construct harboring cDNA encoding Flaveria pringlei H-protein [25] under control of the Solanum tuberosum ST-LS1 promoter [26].

FIG. 2. H-protein overexpressors grow faster and produce more biomass. (A and B) Two individual plants each of the Arabidopsis wild type, FpH L17, and FpH L18 grown side-by-side for six and eight weeks. (C) Rosette diameters, (D) leaf numbers, (E) fresh weight, and (F) dry weight at growth stadium 5.1 [30]. Columns represent mean values±SD (at least 5 individual plants for C, E and F; 25 individual plants for D). Asterisks indicate significant differences to the wild-type control or between lines FpH L17 and L18 (*, p<0.05; **, p<0.01; ***, p<0.001; n.s., not significant). (G) Immunoblots with antibodies against recombinant H-, T-, and P-protein, using 3 μg leaf protein per lane of a denaturing polyacrylamide gel and two plant individuals per overexpressor line. Data for two more lines, FpH L15 and L16, are shown in Supplementary FIG. 2.

FIG. 3. Growth, immunoblots and photosynthetic characteristics of two more H-protein overexpressing lines, FpH L15 and L16, in comparison with lines FpH L17 and L18. (A) Four individual plants each of the Arabidopsis wild type and FpH L15 (left panel) or FpH L16 (right panel) grown side-by-side for six weeks. (B) Immunoblots with antibodies against recombinant H-, T-, and P-protein, using 3 μg leaf protein per lane of a denaturing polyacrylamide gel. Numbers refer to different plants. (C) Net photosynthetic CO2 uptake rates at 400 μL L-1 CO2 and 21% O2. (D) CO2 compensation points at 400 μL L-1 CO2 and 21% O2. Box plots represent mean values±SD (at least 5 individual plants each) for the wild type, FpH L15, and FpH L16 (corresponding values of FpH L17 and FpH L18 from FIG. 2 are included for easier comparison). Asterisks in panels C and D indicate significant differences to the wild-type control (*, p<0.05; **, p<0.01; ***, p<0.001).

FIG. 4. H-protein overexpressors display higher CO2 net-uptake rates, CO2 compensation points and improved light response. (A) Photosynthetic net-CO2 uptake rates at 400 μL L-1 CO2 and 21% O2. (B) CO2 compensation points at 400 μL L-1 CO2 and 21% O2. (C) Relative electron transport rates at varying light intensity in air. Columns and data points represent mean values±SD (at least 5 individual plants per line) for the wild type, FpH L17, and FpH L18. Asterisks indicate significant differences to the wild-type control or between FpH L17 and L18 (*, p<0.05; **, p<0.01; ***, p<0.001; n.s., not significant). Net CO2 uptake rates and CO2 compensation points for two more lines, FpH L15 and L16, are shown in Supplementary FIG. 2.

FIG. 5. H-protein overexpression accelerates the turnover of glycine and RuBP. Relative metabolite contents in leaf samples harvested at mid-day were determined by (A) GC-MS based metabolite profiling [33] and (B) LC-MS based metabolite profiling [34]. Full lists of metabolite changes are shown in Supplementary Tables 1 and 2. Columns represent mean values±SD from at least 4 individual plants. Asterisks indicate significant differences to the wild-type control and between FpH L17 and L18 (*, p<0.05; **, p<0.01; ***, p<0.001; n.s., not significant).

### DETAILED DESCRIPTION OF VARIOUS EMBODIMENTS OF THE INVENTION

The current invention is based on the unexpected finding that facilitating photorespiratory carbon flow improves photosynthetic CO₂ assimilation. Facilitating the photorespiratory carbon flow was achieved by overexpression of the mitochondrial enzyme glycine decarboxylase (GDC). This particular enzyme appeared suitable because it produces the photorespiratory CO₂ [23] and because the leaf glycine level is known as a sensitive indicator of altered photorespiratory carbon flow [24]. Overexpression of the H-protein of glycine decarboxylase considerably enhanced net-photosynthesis and growth of Arabidopsis thaliana. At the molecular level, lower glycine levels confirmed elevated GDC activity in vivo, and lower levels of the CO₂ acceptor ribulose 1,5-bisphosphate indicated higher drain from CO₂ fixation. Thus, the photorespiratory enzyme glycine decarboxylase appears as an important feed-back signaller that contributes to the control of the Calvin-Benson cycle and hence carbon flow through both photosynthesis and photorespiration.

Accordingly, in a first embodiment, the invention provides methods for increasing photosynthesis or photorespiration, or both, in a cell of a plant, in a plant, or in a part of a plant comprising the step of providing a recombinant gene to cells of said plant wherein the recombinant gene comprising the following operably linked DNA fragments:
a. a plant-expressible promoter;
b. a DNA region encoding a subunit of the mitochondrial glycine decarboxylase complex; and
c. optionally, a transcription termination and polyadenylation region.

The mitochondrial glycine decarboxylase complex (GDC, also named glycine-cleavage system or glycine dehydrogenase) is a multi-protein system that occurs in all organisms, prokaryotes and eukaryotes. GDC, together with serine hydroxymethyltransferase (SHMT), is responsible for the inter-conversion of glycine and serine, an essential and ubiquitous step of primary metabolism. In eukaryotes, GDC is present exclusively in the mitochondria, whereas isoforms of SHMT also occur in the cytosol and, in plants, in plastids. The term ‘glycine-serine interconversion’ might suggest that the central importance of this pathway is just the synthesis of serine from glycine and vice versa. However, in both directions of the concerted reaction of GDC and SHMT, tetrahydrofolate (THF) becomes N⁵,N¹⁰-methylenated making these reactions the most important source of active one-carbon-units for a number of biosynthetic processes such as the biosynthesis of methionine, pyrimidines, and purines.

Compared with other organisms, the photorespiratory pathway of plants provides a unique role for both GDC and SHMT. In plants, GDC and SHMT are integral components of primary metabolism not only in the context of ‘house-keeping’ glycine-serine interconversion. Their additional function in plants is the breakdown of glycine that originates, after several enzymatic reactions, from the oxygenase reaction of Rubisco (Bowes et al., 1971; Tolbert, 1973). By this side reaction of oxygenic photosynthesis, 2-phosphoglycolate is produced and, by the action of ten different enzymes including GDC and SHMT, is subsequently recycled as 3-phosphoglycerate to the Calvin cycle.

The course of the reactions in the context of the photorespiratory pathway can be described by the following equations:

### GDC:

Glycine+NAD⁺+THF→Methylene-THF+CO2+NH3+NADH

### SHMT:

Glycine+Methylene-THF+H₂O→Serine+THF

### GDC/SHMT:

2 Glycine+NAD⁺→Serine+CO₂+NH₃+NADH

GDC is a four-protein system comprising three enzymes (P-protein, also known as glycine dehydrogenase [EC 1.4.4.2]; T-protein also known as aminomethyltransferase [EC.2.1.2.10], and L-protein, commonly known as dihydrolipoyl dehydrogenase [EC 1.8.1.4]) plus H-protein, a small lipoylated protein that commutes from one enzyme to the other. First, H-protein conveys the lipoyl-bound aminomethylene intermediate remaining after oxidative glycine decarboxylation from the P- to the T-protein. Eventually, in the reaction catalysed by the L-protein, it donates reducing equivalents to NAD⁺ and becomes re-oxidized.

P protein (EC 1.4.4.2): P protein, a pyridoxal-5-phosphate containing homodimer of about 200 kDa, is the actual glycine decarboxylating subunit. P protein has also been identified as the binding protein of a host-specific toxin victorin. The product of the P protein-catalysed decarboxylation of glycine is CO2 and not bicarbonate. The remaining amino methylene moiety is transferred to the distal sulphur atom of the oxidized lipoamide arm of H protein.

T protein (E.C. 2.1.2.10): T protein, a 45 kDa monomeric aminomethyl transferase, needs THF and H protein as co-substrates. One of the conserved domains of T protein shows significant similarity to a domain of formyltetrahydrofolate synthetase from both prokaryotes and eukaryotes. T protein takes over the aminomethylene group for further processing. The methylene group becomes transferred to tetrahydrofolate resulting in the synthesis of N⁵,N¹⁰-methylene tetrahydrofolate (CH₂-THF) and NH₃ is released. During these reactions, the lipoamide arm of H protein becomes fully reduced and, to be ready for the next cycle, needs to be re-oxidized.

L-protein (EC 1.8.1.4): This reoxidation is achieved by the L-protein (dihydrolipoamide dehydrogenase, LPD). L protein is present as a homodimer of about 100 kDa containing FAD as a coenzyme. During the oxidation of reduced H protein, FAD is reduced to FADH2 which, in turn, becomes immediately reoxidized by NAD+ resulting in the synthesis of one NADH per decarboxylated glycine.

H-protein: H-protein, a 14 kDa lipoamide (5[3-(1,2) dithiolanyl] pentanoic acid) containing non-enzyme protein, interacts as a co-substrate with all three enzyme proteins of the complex. The three-dimensional structures of all forms of H protein have been resolved. Lipoylation of H protein is catalysed by octanoyltransferase in combination with lipoate synthase or by a lipoate-protein ligase and occurs after import of the apoprotein into the mitochondria where lipoic acid is synthesized from fatty acid precursors. Once aminomethylated, the lipoate arm becomes locked within a cleft at the surface of the H protein and released only by interaction with T protein which induces a change in the overall conformation of the H protein. In some plants, tissue-specific alternative splicing results in two H proteins with or without an N-terminal extension of two amino acids. The following protein identifiers can be used to describe and identify the structure of H-proteins: Pfam: PF01597; Pfam clan: CL0105; InterPro: IPR002930; SCOP: 1htp; SUPERFAMILY: 1htp.

In one particular embodiment, the invention provides a method for increasing photosynthesis or photorespiration, or both in a cell of a plant, in a plant, or in part of a plant comprising the step of providing a recombinant gene to cells of said plant, wherein the recombinant gene comprises the following operably linked DNA fragments
a. a plant-expressible promoter;
b. a DNA fragment encoding a mitochondrial glycine decarboxylase lipoylprotein or H-protein; and
c. optionally, a transcription termination and polyadenylation region.

The methods of the invention have been exemplified (see below) using a recombinant gene comprising a cDNA fragment from Flaveria pringlei having the nucleotide sequence of SEQ ID No: 2, which encodes an H-protein comprising the amino acid sequence of SEQ ID No: 1.

However, alternative suitable coding regions for H-proteins may be obtained from other plants such as seed-bearing plants including Aegilops tauschii, Arabidopsis lyrata, Arabidopsis thaliana, Beta vulgaris, Brachypodium distachyon, Cicer arietinum, Cucumis sativus, Flaveria anomala, Flaveria bidentis, Flaveria brownii, Flaveria chlorifolia, Flaveria cronquistii, Flaveria floridana, Flaveria linearis, Flaveria palmeri, Flaveria pringlei, Flaveria pubescens, Flaveria trinervia, Glycine max, Hordeum vulgare subsp. vulgare, Lotus japonica, Medicago truncatula, Oryza sativa Indica Group, Oryza sativa Japonica Group, Pinus pinaster, Pisum abyssinicum, Pisum fulvum, Pisum sativum subsp. elatius, Pisum sativum subsp. transcaucasicum, Pisum sativum var. pumilio, Pisum sativum var. tibetanicum, Pisum sativum, Populus tremuloides, Populus trichocarpa, Ricinus communis, Sonneratia alba, Sorghum bicolor, Sorghum bicolor, Triticum aestivum, Triticum urartu, Vitis vinifera or Zea mays. Suitable coding regions for H-proteins may also be obtained from green algae, including Micromonas or Chlamydomonas.

Different amino acid sequences for H-proteins from plants are known in the art and available from databases such as the protein sequences identified by the following accession numbers: (Flaveria trinervia) Accession: CAA85760.1-GI: 547502; (Flaveria anomala) Accession: CAA85761.1-GI:547558; (Flaveria pringlei) Accession: CAA85759.1-GI: 547500; (Flaveria pringlei) Accession: CAA81075.1-GI: 438001; (Flaveria pringlei) Accession: CAA81074.1-GI: 437999; (Pisum sativum) Accession: CAA37704.1-GI: 287815; (Flaveria cronquistii) Accession: CAA85756.1-GI: 547521; (Flaveria cronquistii) Accession: CAA85755.1-GI: 547519; (Flaveria cronquistii) Accession: CAA81073.1-GI: 437993; (Flaveria pubescens) Accession: CAA85768.1-GI: 547564; (Flaveria palmeri) Accession: CAA85767.1-GI: 547562; (Flaveria floridana) Accession: CAA85766.1-GI: 547560; (Flaveria linearis) Accession: CAA85758.1-GI: 547498; (Flaveria chlorifolia) Accession: CAA85757.1-GI: 547496; (Flaveria bidentis) Accession: CAA85754.1-GI: 547494; (Pisum sativum) Accession: CAA45978.1-GI: 20737; (Flaveria brownii) Accession: CAA94317.1-GI: 1240038; (Flaveria trinervia) Accession: CAA94316.1-GI: 1240036; (Pisum fulvum) Accession: CAI79404.1-GI: 62700762; (Pisum sativum) Accession: 1HPC_B-GI: 1065302; (Pisum sativum) Accession: 1HPC_A-GI: 106530; (Pisum sativum) Accession: 1DXM_B-GI: 9955326; (Pisum sativum) Accession: 1DXM_A-GI: 9955325; (Pisum sativum) Accession: AAA33668.1-GI: 169093; (Pisum sativum) Accession: 1HTP_A-GI: 157831395; (Arabidopsis thaliana) Accession: AAA87942.1-GI: 861215; (Populus trichocarpa) Accession: EEF07364.1-GI: 222870233; (Populus trichocarpa) Accession: EEF05946.1-GI: 222868815; (Populus trichocarpa) Accession: EEE97119.1-GI: 222859572; (Populus trichocarpa) Accession: EEE72509.1-GI: 222834032; (Populus tremuloides) Accession: AAO63775.1-GI: 29124971; (Populus trichocarpa) Accession: XP_002326710.1-GI: 224138868; (Populus trichocarpa) Accession: XP_002321819.1-GI: 224134418; (Populus trichocarpa) Accession: XP_002329524.1-GI: 224126315; (Populus trichocarpa) Accession: XP_002318899.1-GI: 224122680; (Populus tremuloides) Accession: AAQ67414.2-GI: 118430834; (Populus tremuloides) Accession: ABO61731.1-GI: 134142794; (Populus tremuloides) Accession: ABJ98947.1-GI: 116490125; (Oryza sativa Japonica Group) Accession: BAD45416.1-GI: 52076539; (Oryza sativa Japonica Group) Accession: BAD45431.1-GI: 52075823; (Pinus pinaster) Accession: CCC55429.1-GI: 346983241; (Pinus pinaster) Accession: CCC55419.1-GI: 346453264; (Sonneratia alba) Accession: ACS68725.1-GI: 241865386; (Sonneratia alba) Accession: ACS68655.1-GI: 241865154; (Arabidopsis thaliana) Accession: AAM64413.1-GI: 21592462; (Arabidopsis thaliana) Accession: AAC61829.1-GI: 3668097; (Arabidopsis thaliana) Accession: AAC36184.1-GI: 3608151; (Oryza sativa Japonica Group) Accession: BAD25184.1-GI: 49388072; (Oryza sativa Japonica Group) Accession: BAD25486.1-GI: 49387555; (Pisum abyssinicum) Accession: CAJ13736.1-GI: 68609794; (Pisum abyssinicum) Accession: CAJ13735.1-GI: 68609789; (Pisum abyssinicum) Accession: CAJ13734.1-GI: 68609784; (Pisum abyssinicum) Accession: CAJ13733.1-GI: 68609776; (Pisum sativum subsp. elatius) Accession: CAJ13732.1-GI: 68609771; (Pisum sativum subsp. elatius) Accession: CAJ13731.1-GI: 68609766; (Pisum sativum subsp. elatius) Accession: CAJ13730.1-GI: 68609761; (Pisum sativum subsp. elatius) Accession: CAJ13729.1-GI: 68609757; (Pisum sativum) Accession: CAJ13726.1-GI: 68609743; (Pisum sativum) Accession: CAJ13725.1-GI: 68609738; (Pisum sativum) Accession: CAJ13724.1-GI: 68609733; (Pisum sativum var. pumilio) Accession: CAJ13723.1-GI: 68609728; (Pisum sativum var. pumilio) Accession: CAJ13722.1-GI: 68609723; (Pisum sativum var. pumilio) Accession: CAJ13721.1-GI: 68609718; (Arabidopsis thaliana) Accession: BAE99735.1-GI: 110743799; (Arabidopsis thaliana) Accession: BAF00389.1-GI: 110736863; (Oryza sativa Japonica Group) Accession: AAG13505.2-GI: 10257441; (Oryza sativa Indica Group) Accession: AAB82134.1-GI: 2570497; (Pisum fulvum) Accession: CAJ13728.1-GI: 68609752; (Pisum fulvum) Accession: CAJ13727.1-GI: 68609748; (Flaveria trinervia) Accession: P46485.1-GI: 1169884; (Arabidopsis thaliana) Accession: AEC09100.1-GI: 330254006; (Arabidopsis thaliana) Accession: NP_181080.1-GI: 15226973; (Arabidopsis thaliana) Accession: AEE31490.1-GI: 332193369; (Arabidopsis thaliana) Accession: AEC09067.1-GI: 330253973; (Arabidopsis thaliana) Accession: NP_181057.1-GI: 15226906; (Arabidopsis thaliana) Accession: NP_174525.1-GI: 15223217; (Oryza sativa Japonica Group) Accession: AAK39594.1-GI: 13786469; (Triticum aestivum) Accession: AAM92707.1-GI: 22204118; (Cicer arietinum) Accession: AEP95748.1-GI: 349592193; (Cicer arietinum) Accession: AEP95744.1-GI: 349592185; (Pisum sativum) Accession: CAJ13840.1-GI: 68638237; (Pisum sativum) Accession: CAJ13839.1-GI: 68638233; (Pisum sativum subsp. transcaucasicum) Accession: CAJ13838.1-GI: 68638228: (Pisum sativum subsp. transcaucasicum) Accession: CAJ13837.1-GI: 68638223; (Pisum sativum var. tibetanicum) Accession: CAJ13836.1-GI: 68638215; (Pisum sativum) Accession: CAJ13835.1-GI: 68638210; (Pisum sativum) Accession: CAJ13834.1-GI: 68638203; (Pisum sativum subsp. asiaticum) Accession: CAJ13833.1-GI: 68638194; (Pisum sativum) Accession: CAJ13832.1-GI: 68638189; (Pisum sativum) Accession: CAJ13831.1-GI: 68638183; (Pisum sativum) Accession: CAJ13830.1-GI: 68638175; (Pisum sativum) Accession: CAJ13829.1-GI: 68638168; (Pisum sativum) Accession: CAJ13828.1-GI: 68638161; (Pisum fulvum) Accession: CAJ13415.1-GI: 68609706; (Pisum sativum) Accession: CAA38252.1-GI: 20739; (Beta vulgaris) Accession: AAL04441.1-GI: 15637149; (Pisum sativum) Accession: P16048.1-GI: 121080; (Arabidopsis thaliana) Accession: P25855.1-GI: 121075; (Arabidopsis thaliana) Accession: Q9LQL0.1-GI: 12644523; (Arabidopsis thaliana) Accession: 082179.1-GI: 75220222; (Flaveria pubescens) Accession: P49360.1-GI: 1346119; (Flaveria pringlei) Accession: P49359.1-GI: 1346118; (Arabidopsis thaliana) Accession: AAA32802.1-GI: 166725; (Oryza sativa Japonica Group) Accession: BAG92586.1-GI: 215701162; (Oryza sativa Japonica Group) Accession: BAF20229.1-GI: 113596355; (Oryza sativa Japonica Group) Accession: NP_001058315.1-GI: 115469432; (Sorghum bicolor) Accession: EES04594.1-GI: 241931449; (Arabidopsis thaliana) Accession: AAM19865.1-GI: 20453253; (Arabidopsis thaliana) Accession: AAL31106.1-GI: 16974361; (Arabidopsis thaliana) Accession: AAL24242.1-GI: 16604472; (Arabidopsis thaliana) Accession: AAL06993.1-GI: 15810184; (Arabidopsis thaliana) Accession: AAK91461.1-GI: 15215833; (Sorghum bicolor) Accession: XP_002451618.1-GI: 242060658; (Arabidopsis thaliana) Accession: 1908425A-GI: 445119; (Medicago truncatula) Accession: ACJ85876.1-GI:217075032; (Medicago truncatula) Accession: AFK47219.1-GI:388518315; (Medicago truncatula) Accession: AES94422.1-GI:355512799; (Medicago truncatula) Accession: XP_003611464.1 GI:357482357; (Lotus japonica) Accession: AFK42846.1-GI:388509560; (Vitis vinifera) Accession: CBI33899.3-GI:297742112; (Vitis vinifera) Accession: XP_002280707.1-GI:225427234; (Vitis vinifera) Accession: CAN76620.1-GI:147770018; (Cucumis sativus) Accession: XP_004164375.1-GI:449513621; (Cucumis sativus) Accession: XP_004148493.1-GI:449461527; (Glycine max) Accession: XP_003539169.1-GI:356541408; (Ricinus communis) Accession: XP_002519845.1-GI:255557631; (Ricinus communis) Accession: EEF42449.1-GI:223540891; (Glycine max) Accession: ACU14940.1-GI:255629191; (Arabidopsis lyrata) Accession: EFH69985.1-GI:297339568; (Brachypodium distachyon) Accession: XP_003574175.1-GI:357146969; (Hordeum vulgare subsp. vulgare) Accession: BAJ95506.1-GI:326505670; (Hordeum vulgare subsp. vulgare) Accession: BAK03631.1-GI:326515436; (Hordeum vulgare subsp. vulgare) Accession: BAJ88062.1-GI:326516078; (Aegilops tauschii) Accession: EMT30735.1-GI:475619314; (Triticum urartu) Accession: EMS55121.1-GI: 474097201; (Zea mays) Accession: AAL33596.1-GI:17017277; (Zea mays) Accession: NP_001141253.1-GI:226533407; (Zea mays) Accession: ACF85859.1-GI:194703550; (Glycine max) Accession: NP_001242407.1-GI:363807578; (Glycine max) Accession: ACU13688.1-GI:255626687 (herein incorporated by reference).

Different sequences for H-proteins from green algae are known in the art and available from databases such as the protein sequences identified by the following accession numbers: (Micromonas sp. RCC299) Accession: ACO61937.1-GI: 226515942; (Micromonas pusilla CCMP1545) Accession: EEH51265.1-GI: 226453958; (Chlamydomonas reinhardtii); Accession: EDP08614.1-GI: 158282862; (Micromonas pusilla CCMP1545) Accession: XP_003064360.1-GI: 303290146; (Micromonas sp. RCC299) Accession: XP_002500679.1-GI: 255074009; (Chlamydomonas reinhardtii) Accession: XP_001696637.1-GI: 159477076; (Chlamydomonas incerta) Accession: ABA01127.1-GI: 74272663; (Chlamydomonas incerta) Accession: AAV71155.1-GI: 56112390; (Chlamydomonas reinhardtii) Accession: AAK70873.1-GI: 14595650 (herein incorporated by reference).

It will be clear that nucleotide sequence encoding variants of H-proteins, wherein one or more amino acid residues have been deleted, substituted or inserted, which can be deduced from the above mentioned amino acid sequences, can also be used to the same effect in the methods according to the invention, provided that the H-protein variant can still serve as a substrate for P-, T- and L-protein. Glycine decarboxylase enzymatic activity assays are known in the art and have been described e.g. by Laywer and Zelitch (1979) Plant Physiol. 64, 706-711.

Moreover, DNA fragments encoding H-proteins, may also be made synthetically, even with a codon usage adapted to the preferred codon-usage of the plant in which the recombinant gene can be introduced.

Other DNA fragments suitable for methods according to the invention are DNA fragments that hybridize under stringent conditions with the above mentioned DNA fragments encoding H-proteins. The terms “stringent conditions” or “stringent hybridization conditions” include reference to conditions under which a probe will hybridize to its target sequence, to a detectably greater degree than other sequences (e.g., at least 2-fold over background). Stringent conditions are sequence-dependent and will be different in different circumstances. By controlling the stringency of the hybridization and/or washing conditions, target sequences can be identified which can be up to 100% complementary to the probe (homologous probing). Alternatively, stringency conditions can be adjusted to allow some mismatching in sequences so that lower degrees of similarity are detected (heterologous probing). Optimally, the probe is approximately 500 nucleotides in length, but can vary greatly in length from less than 500 nucleotides to equal to the entire length of the target sequence. Typically, stringent conditions will be those in which the salt concentration is less than about 1.5 M Na ion, typically about 0.01 to 1.0 M Na ion concentration (or other salts) at pH 7.0 to 8.3 and the temperature is at least about 300 C for short probes (e.g., 10 to 50 nucleotides) and at least about 600 C for long probes (e.g., greater than 50 nucleotides). Stringent conditions may also be achieved with the addition of destabilizing agents such as formamide or Denhardt's. Exemplary low stringency conditions include hybridization with a buffer solution of 30 to 35% formamide, 1 M NaCl, 1% SDS at 37° C., and a wash in 1× to 2×SSC at 50 to 55° C. Exemplary moderate stringency conditions include hybridization in 40 to 45% formamide, 1 M NaCl, 1% SDS at 37° C. and a wash in 0.5X to 1×SSC at 55 to 600 C. Exemplary high stringency conditions include hybridization in 50% formamide, 1 M NaCl, 1% SDS at 37° C. and a wash in 0.1×SSC at 60 to 65° C. Specificity is typically the function of post-hybridization washes, the critical factors being the ionic strength and temperature of the final wash solution. For DNA-DNA hybrids, the Tm can be approximated from the equation of Meinkoth and Wahl, (1984) Anal. Biochem., 138:267-84: Tm=81.5° C.+16.6 (log M)+0.41 (% GC)−0.61 (% form)−500/L; where M is the molarity of monovalent cations, % GC is the percentage of guanosine and cytosine nucleotides in the DNA, % form is the percentage of formamide in the hybridization solution, and L is the length of the hybrid in base pairs. The Tm is the temperature (under defined ionic strength and pH) at which 50% of a complementary target sequence hybridizes to a perfectly matched probe. Tm is reduced by about 1° C. for each 1% of mismatching; thus, Tm, hybridization and/or wash conditions can be adjusted to hybridize to sequences of the desired identity. For example, if sequences with >90% identity are sought, the Tm can be decreased 100 C. Generally, stringent conditions are selected to be about 5° C. lower than the thermal melting point (Tm) for the specific sequence and its complement at a defined ionic strength and pH. However, severely stringent conditions can utilize a hybridization and/or wash at 1, 2, 3 or 4° C. lower than the thermal melting point (Tm); moderately stringent conditions can utilize a hybridization and/or wash at 6, 7, 8, 9 or 100 C lower than the thermal melting point (Tm); low stringency conditions can utilize a hybridization and/or wash at 11, 12, 13, 14, 15 or 200 C lower than the thermal melting point (Tm). Using the equation, hybridization and wash compositions and desired Tm, those of ordinary skill will understand that variations in the stringency of hybridization and/or wash solutions are inherently described. If the desired degree of mismatching results in a Tm of less than 45° C. (aqueous solution) or 32° C. (formamide solution) it is preferred to increase the SSC concentration so that a higher temperature can be used. An extensive guide to the hybridization of nucleic acids is found in Tijssen, Laboratory Techniques in Biochemistry and Molecular Biology—Hybridization with Nucleic Acid Probes, part I, chapter 2, “Overview of principles of hybridization and the strategy of nucleic acid probe assays,” Elsevier, New York (1993); and Current Protocols in Molecular Biology, chapter 2, Ausubel, et al., eds, Greene Publishing and Wiley-Interscience, New York (1995). Unless otherwise stated, in the present application high stringency is defined as hybridization in 4×SSC, 5×Denhardt's (5 g Ficoll, 5 g polyvinypyrrolidone, 5 g bovine serum albumin in 500 ml of water), 0.1 mg/ml boiled salmon sperm DNA and 25 mM Na phosphate at 65° C. and a wash in 0.1×SSC, 0.1% SDS at 65° C.

Other DNA fragments suitable for methods according to the invention are DNA fragments encoding a polypeptide having an amino acid sequence sharing at least 80%, 81%, 82%, 83%, 84%, 85%, 86%, 87%, 88%, 89%, 90%, 91%, 92%, 93%, 94%, 95%, 96%, 97%, 98%, 99% or 100% sequence identity with any of the above mentioned amino acid sequences of H-proteins, or that comprise a nucleotide sequence having at least 80%, 81%, 82%, 83%, 84%, 85%, 86%, 87%, 88%, 89%, 90%, 91%, 92%, 93%, 94%, 95%, 96%, 97%, 98%, 99% or 100% sequence identity with any of the above mentioned nucleotide sequences encoding H-proteins.

For the purpose of this invention, the “sequence identity” of two related nucleotide or amino acid sequences, expressed as a percentage, refers to the number of positions in the two optimally aligned sequences which have identical residues (×100) divided by the number of positions compared. A gap, i.e. a position in an alignment where a residue is present in one sequence but not in the other is regarded as a position with non-identical residues. The alignment of the two sequences is performed by the Needleman and Wunsch algorithm (Needleman and Wunsch 1970) Computer-assisted sequence alignment, can be conveniently performed using standard software program such as GAP which is part of the Wisconsin Package Version 10.1 (Genetics Computer Group, Madison, Wis., USA) using the default scoring matrix with a gap creation penalty of 50 and a gap extension penalty of 3.

It will be clear that whenever nucleotide sequences of RNA molecules are defined by reference to nucleotide sequence of corresponding DNA molecules, the thymine (T) in the nucleotide sequence should be replaced by uracil (U). Whether reference is made to RNA or DNA molecules will be clear from the context of the application.

As used herein, the term “promoter” denotes any DNA which is recognized and bound (directly and indirectly) by a DNA-dependent RNA-polymerase during initiation of transcription. A promoter includes the transcription initiation site, and binding sites for transcription initiation factors and RNA polymerase, and can comprise various other sites (e.g. enhancers), at which gene expression regulatory proteins may bind.

As used herein, a “plant expressible promoter” is a promoter capable of functioning in plant cells and plants. Examples include bacterial promoters, such as that of octopine synthase (OCS) and nopaline synthase (NOS) promoters from Agrobacterium, but also viral promoters, such as that of the cauliflower mosaic virus (CaMV) 35S or 19S RNAs genes (Odell et al., 1985, Nature. 6; 313(6005):810-2), promoters of the cassava vein mosaic virus (CsVMV; WO 97/48819), the sugarcane bacilliform badnavirus (ScBV) promoter (Samac et al., 2004, Transgenic Res. 13(4):349-61), the figwort mosaic virus (FMV) promoter (Sanger et al., 1990, Plant Mol Biol. 14(3):433-43) and the subterranean clover virus promoter No 4 or No 7 (WO 96/06932). Among the promoters of plant origin, mention will be made of the promoters of the Rubisco small subunit promoter (U.S. Pat. No. 4,962,028), the ubiquitin promoters of Maize, Rice and sugarcane, the Rice actin 1 promoter (Act-1) and the Maize alcohol dehydrogenase 1 promoter (Adh-1).

The methods of the invention have been exemplified using a leaf-specific and light regulated Solanum tuberosum ST-LS1 promoter (Stockhaus et al. 1989, EMBO J. 8, 2445-2451) also represented herein as SEQ ID No: 3 from nucleotide 1 to nucleotide 1571.

However, it will be immediately clear that an alternative light-regulated promoter can be used to the same effect. Light-inducible plant-expressible promoters suitable for the invention may include the following promoters:
a) promoters from genes encoding small subunit of ribulose-1,5-biphosphate carboxylase/oxygenase (rbcS) such as the rbcS gene from Coffea arabica, Accession: AJ419827.1-GI: 24940139; Lemna gibba, Accession: FJ626428.1-GI: 223018280; Zea mays, Accession: AH005359.3-GI: 339635306; Pisum sativum, Accession: DQ141599.1-GI: 74058522; Oryza sativa (japonica cultivar-group), Accession: AY583764.1-GI: 46982178; Lactuca sativa, Accession: JQ741945.1-GI: 384875920; Gossypium hirsutum, Accession: DQ648074.1-GI: 109644667; Malus×domestica, Accession: HM222640.1-GI: 307547080; Malus×domestica, Accession: HM222639.1-GI: 307547079; Zea mays, Accession: S42508.1-GI: 253496; Brassica napus, Accession: X75334.1-GI: 406726; Zea mays, Accession: S42568.1-GI: 253497; Pisum sativum, Accession: M21356.1-GI: 169149; Lemna gibba, Accession: S45167.1-GI: 257044; Lemna gibba, Accession: S45166.1-GI: 257043; Lemna gibba, Accession: S45165.1-GI: 257042; Arabidopsis thaliana, Accession: AB196447.1-GI: 56550547; Lycopersicon esculentum Accession: S44160.1-GI: 255571;
b) promoters from chlorophyll ab/b binding protein encoding genes (Lhc, formerly called Cab) such as the Lhc from Zea mays, Accession: M87020.1-GI: 168438; Arabidopsis thaliana, Accession: AB196448.1-GI: 56550548; Beta vulgaris, Accession: AJ579711.2-GI: 33504459; Pisum sativum, Accession: X03074.1-GI: 20629; Brassica napus, Accession: X61609.1-GI: 405614; Glycine max, Accession: X12981.1-GI: 18551; Glycine max, Accession: X12980.1-GI: 18547; Zea mays Accession: M87020.1-GI: 168438; Malus×domestica, Accession: X17697.1-GI: 19540; Petunia, Accession: X02356.1-GI: 20486; Petunia, Accession: X02358.1-GI: 20482; Petunia, Accession: X02360.1-GI: 20478; Petunia, Accession: X02359.1-GI: 20474; Petunia, Accession: X02357.1-GI: 20470; Hordeum vulgare, Accession: X12735.1-GI: 18942; Prunus persica, Accession: EF127291.1-GI: 126508509; Oryza sativa, Accession: NC_008397.2-GI: 297603645; Brassica napus, Accession: X61608.1-GI: 515615; Oryza sativa, Accession: X13908.1-GI:20177; Zea mays, Accession: X14794.1-GI: 22223; Oryza sativa, Accession: X13909.1-GI: 20181; Brassica juncea, Accession: X16436.1-GI: 21137;
c) promoters including light regulatory elements (Bruce and Quaill, Plant Cell 2 (11):1081-1089 (1990); Bruce et al., EMBO J. 10:3015-3024 (1991); Rocholl et al, Plant Sci. 97:189-198 (1994); Block et al, Proc. Natl. Acad. Sci. USA 87:5387-5391 (1990); Giuliano et al, Proc. Natl. Acad. Sci. USA 85:7089-7093 (1988); Staiger et al., Proc. Natl. Acad. Sci USA 86:6930-6934 (1989); Izawa et al., Plant Cell 6:1277-1287 (1994); Menkens et al, Trends in Biochemistry 20:506-510 (1995); Foster et al, FASEB J. 8:192-200 (1994); Plesse et al, Mol. Gen. Genet. 254:258-266 (1997); Green et al, EMBO J. 6:2543-2549 (1987); Kuhlemeier et al, Ann. Rev Plant Physiol. 38:221-257 (1987); Villain et al, J. Biol. Chem. 271:32593-32598 (1996); Lam et al, Plant Cell 2:857-866 (1990); Gilmartin et al, Plant Cell 2:369-378 (1990); Datta et al, Plant Cell 1: 1069-1077 (1989); Gilmartin et al, Plant Cell 2:369-378 (1990); Castresana et al, EMBO J. 7:1929-1936 (1988); Ueda et al, Plant Cell 1:217-227 (1989); Terzaghi et al, Annu Rev. Plant Physiol. Plant MoI Biol. 46:445-474 (1995); Green et al, EMBO J. 6:2543-2549 (1987); Villain et al, J. Biol. Chem. 271:32593-32598 (1996); Tjaden et al, Plant Cell 6:107-118 (1994); Tjaden et al, Plant Physiol. 108:1109-1117 (1995); Ngai et al, Plant J. 12:1021-1234 (1997); Bruce et al, EMBO J. 10:3015-3024 (1991); Ngai et al, Plant J. 12: 1021-1034 (1997);
d) promoters of the light-inducible transcripts described in WO 2010/138328, particularly in Table 1 on pages 18 and 19 and included in the sequence listing of that application as SEQ ID Nos. 1 to 17 (incorporated herein by reference).

In one embodiment, the light-inducible promoter is also a promoter preferentially expressed, or selectively expressed in green tissues. In another embodiment, the light-inducible promoter is also a promoter preferentially expressed or selectively expressed in the mesophyll. In yet another embodiment of the invention, the light-inducible promoter is preferentially or selectively expressed in green tissues and mesophyll. As used herein, “preferentially expressed” indicates that the promoter directs transcription of an operably linked DNA fragment to a higher extent in the mentioned tissues than in the rest of the plant. “Selectively expressed” indicates that the promoter directs transcription of an operably linked DNA fragment to a significantly higher extent in the mentioned tissues than in the rest of the plant, including embodiments where the promoter is only very low expressed (relative vs the preferred tissues) in other tissues or even not expressed for all practical intents and purposes.

The term “transcription termination and polyadenylation region” encompasses a control sequence which is a DNA sequence at the end of a transcriptional unit which signals 3′ processing and polyadenylation of a primary transcript and termination of transcription. The terminator can be derived from the natural gene, from a variety of other plant genes, from viral genes (CaMV 35 terminator) or from T-DNA genes. The terminator to be added may be derived from, for example, the nopaline synthase or octopine synthase genes, or alternatively from another plant gene, or less preferably from any other eukaryotic gene. The terminator should be functional in cells of a plant.

Having read the above mentioned methods according to the invention, a person skilled in the art will realize that similar effects can be achieved by increasing the carbon flow through the photorespiration utilizing the other subunits of the glycine cleavage system, i.e. by increasing the expression of the P, T or L-protein.

The obtained plants comprising a recombinant gene according to the invention grow faster and have an increased biomass when compared to isogenic plants not containing the recombinant gene. Accordingly, in another embodiment of the invention, a method is provided to increase yield, growth or biomass (or both) of a plant comprising the step of providing the cells of the plant with a recombinant gene wherein the recombinant gene comprises operably linked:
a) a light-inducible plant-expressible promoter; including a light-inducible promoter preferentially or selectively expressed in green tissue and/or mesophyll.
b) a DNA region encoding a subunit of the mitochondrial glycine decarboxylase complex such as the mitochondrial H-protein as herein elsewhere described; and
c) optionally, a 3′ end region involved in transcription termination and polyadenylation, preferably a 3′ end region functional in plant cells.

As used herein “yield” generally refers to a measurable produce from a plant, particularly a crop. Yield and yield increase (in comparison to a non-transformed isogenic plant) can be measured in a number of ways, and a skilled person will be able to apply the correct meaning of the term yield in the context of the particular crop concerned and the specific purpose or application concerned.

As used herein, the term “improved yield” or the term “increased yield” means any improvement in the yield of any measured plant product, such as grain, fruit or fiber or biomass. Parameters such as floral organ development, root initiation, root biomass, seed number, seed weight, harvest index, leaf formation and fruit development, are suitable measurements of improved yield. The improvement in yield can comprise a 0.1%, 0.5%, 1%, 3%, 5%, 10%, 15%, 20%, 30%, 40%, 50%, 60%, 70%, 80%, 90% or greater increase in any measured parameter. Yield may also refers to biomass yield, dry biomass yield, aerial dry biomass yield, underground dry biomass yield, fresh-weight biomass yield, aerial fresh-weight biomass yield, underground fresh-weight biomass yield; enhanced yield of harvestable parts, enhanced yield of crop fruit, enhanced yield of seeds.

Crop yield is defined herein as the number of bushels of relevant agricultural product (such as grain, forage, or seed) harvested per acre or tons per hectare. Yield can be calculated as harvest index (expressed as a ratio of the weight of the respective harvestable parts divided by the total biomass), harvestable parts weight per area (acre, square meter, or the like); and the like. Yield may also be calculated on a per plant basis. Yield may also refer to seed yield which can be measured by one or more of the following parameters: number of seeds or number of filled seeds (per plant or per area (acre/square meter/or the like)); seed filling rate (ratio between number of filled seeds and total number of seeds); number of flowers per plant; seed biomass or total seeds weight (per plant or per area (acre/square meter/or the like); thousand kernel weight (TKW; extrapolated from the number of filled seeds counted and their total weight; an increase in TKW may be caused by an increased seed size, an increased seed weight, an increased embryo size, and/or an increased endosperm). Seed yield may be determined on a dry weight or on a fresh weight basis, or typically on a moisture adjusted basis.

Increased yield for corn plants may mean in one embodiment, increased seed yield, in particular for corn varieties used for feed or food. Also in soybean, rice, wheat, cereal crops or oilseed rape, a relevant yield parameter is increased seed yield, in particular for soy varieties used for feed or food. In other crops, such as cotton, flax, hennep and other fiber-producing plants, Increased yield may refer to increase fiber yield, and for cotton specifically increased lint yield.

The methods of the invention require that a recombinant gene be provided to the cells of a plant. As used herein “providing” encompasses introduction a recombinant gene into cells of a plant via crossing with a plant already comprising such recombinant gene and selection of the appropriate progeny plants. The recombinant gene may also be provided to plant cells in alternative ways, e.g. via protoplast fusion between a cell comprising the recombinant gene and a target cell. Providing a recombinant gene also encompasses introduction of a recombinant gene via transformation, either stably or transiently. Transformation of plant species is well known in the art. Advantageously, any of several transformation methods may be used to introduce the recombinant gene into a suitable ancestor cell. The methods described for the transformation and regeneration of plants from plant tissues or plant cells may be utilized for transient or for stable transformation. Transformation methods include the use of liposomes, electroporation, chemicals that increase free DNA uptake, injection of the DNA directly into the plant, particle gun bombardment, transformation using viruses or pollen and microprojection. Methods may be selected from the calcium/polyethylene glycol method for protoplasts (Krens, F. A. et al., (1982) Nature 296, 72-74; Negrutiu I et al. (1987) Plant Mol Biol 8: 363-373); electroporation of protoplasts (Shillito R. D. et al. (1985) Bio/Technol 3, 1099-1102); microinjection into plant material (Crossway A et al., (1986) Mol. Gen Genet 202: 179-185); DNA or RNA-coated particle bombardment (Klein T M et al., (1987) Nature 327: 70) infection with (non-integrative) viruses and the like. Transgenic plants, including transgenic crop plants, are preferably produced via Agrobacterium-mediated transformation. Methods for Agrobacterium-mediated transformation of rice include those described by Hiei et al. (Plant J 6 (2): 271-282, 1994). In the case of corn transformation, a suitable method is as described in Ishida et al. (Nat. Biotech. 14(6): 745-50, 1996). Other methods are described in B. Jenes et al., Techniques for Gene Transfer, in: Transgenic Plants, Vol. 1, Engineering and Utilization, eds. S. D. Kung and R. Wu, Academic Press (1993) 128-143. The transformation of the chloroplast genome is generally achieved by a process which has been schematically displayed in Klaus et al., 2004 [Nature Biotechnology 22 (2), 225-229]. Briefly the sequences to be transformed are cloned together with a selectable marker gene between flanking sequences homologous to the chloroplast genome. These homologous flanking sequences direct site specific integration into the plastome. Plastidal transformation has been described for many different plant species and an overview is given in Bock (2001) Transgenic plastids in basic research and plant biotechnology. J Mol Biol. 2001 Sep. 21; 312 (3):425-38 or Maliga, P (2003) Progress towards commercialization of plastid transformation technology. Trends Biotechnol. 21, 20-28.

The methods of the invention are useful in any plant. Preferred plants are seed-bearing plants, including gymnosperms and angiosperms, particularly monocotyledonous or dicotyledonous plants, including from oilseed rape, cotton, rice, soybean, wheat, sugarcane or corn, but also vegetables, fiber-producing plants, shrubs and trees, grasses, small grain cereals and the like.

The methods may be applied to a plant is selected from Acer spp., Actinidia spp., Abelmoschus spp., Agave sisalana, Agropyron spp., Agrostis stolonifera, Allium spp., Amaranthus spp., Ammophila arenaria, Ananas comosus, Annona spp., Apium graveolens, Arachis spp, Artocarpus spp., Asparagus officinalis, Avena spp. (e.g. Avena sativa, Avena fatua, Avena byzantina, Avena fatua var. sativa, Avena hybrida), Averrhoa carambola, Bambusa sp., Benincasa hispida, Bertholletia excelsea, Beta vulgaris, Brassica spp. (e.g. Brassica napus, Brassica rapa ssp. [canola, oilseed rape, turnip rape]), Cadaba farinosa, Camellia sinensis, Canna indica, Cannabis sativa, Capsicum spp., Carex elata, Carica papaya, Carissa macrocarpa, Carya spp., Carthamus tinctorius, Castanea spp., Ceiba pentandra, Cichorium endivia, Cinnamomum spp., Citrullus lanatus, Citrus spp., Cocos spp., Coffea spp., Colocasia esculenta, Cola spp., Corchorus sp., Coriandrum sativum, Corylus spp., Crataegus spp., Crocus sativus, Cucurbita spp., Cucumis spp., Cynara spp., Daucus carota, Desmodium spp., Dimocarpus longan, Dioscorea spp., Diospyros spp., Echinochloa spp., Elaeis (e.g. Elaeis guineensis, Elaeis oleifera), Eleusine coracana, Eragrostis tef Erianthus sp., Eriobotrya japonica, Eucalyptus sp., Eugenia un flora, Fagopyrum spp., Fagus spp., Festuca arundinacea, Ficus carica, Fortunella spp., Fragaria spp., Ginkgo biloba, Glycine spp. (e.g. Glycine max, Soja hispida or Soja max), Gossypium hirsutum, Gossypium barbadense, Helianthus spp. (e.g. Helianthus annuus), Hemerocallis fulva, Hibiscus spp., Hordeum spp. (e.g. Hordeum vulgare), Ipomoea batatas, Juglans spp., Lactuca sativa, Lathyrus spp., Lens culinaris, Linum usitatissimum, Litchi chinensis, Lotus spp., Luffa acutangula, Lupinus spp., Luzula sylvatica, Lycopersicon spp. (e.g. Lycopersicon esculentum, Lycopersicon lycopersicum, Lycopersicon pyriforme), Macrotyloma spp., Malus spp., Malpighia emarginata, Mammea americana, Mangifera indica, Manihot spp., Manilkara zapota, Medicago sativa, Melilotus spp., Mentha spp., Miscanthus sinensis, Momordica spp., Morus nigra, Musa spp., Nicotiana spp., Olea spp., Opuntia spp., Ornithopus spp., Oryza spp. (e.g. Oryza sativa, Oryza latifolia), Panicum miliaceum, Panicum virgatum, Passiflora edulis, Pastinaca sativa, Pennisetum sp., Persea spp., Petroselinum crispum, Phalaris arundinacea, Phaseolus spp., Phleum pratense, Phoenix spp., Phragmites australis, Physalis spp., Pinus spp., Pistacia vera, Pisum spp., Poa spp., Populus spp., Prosopis spp., Prunus spp., Psidium spp., Punica granatum, Pyrus communis, Quercus spp., Raphanus sativus, Rheum rhabarbarum, Ribes spp., Ricinus communis, Rubus spp., Saccharum spp., Salix sp., Sambucus spp., Secale cereale, Sesamum spp., Sinapis sp., Solanum spp. (e.g. Solanum tuberosum, Solanum integrifolium or Solanum lycopersicum), Sorghum bicolor, Spinacia spp., Syzygium spp., Tagetes spp., Tamarindus indica, Theobroma cacao, Trifolium spp., Tripsacum dactyloides, Triticosecale rimpaui, Triticum spp. (e.g. Triticum aestivum, Triticum durum, Triticum turgidum, Triticum hybernum, Triticum macha, Triticum sativum, Triticum monococcum or Triticum vulgare), Tropaeolum minus, Tropaeolum majus, Vaccinium spp., Vicia spp., Vigna spp., Viola odorata, Vitis spp., Zea mays, Zizania palustris, Ziziphus spp. amongst others.

In another embodiment, the invention also provides plant cells, plants, plant parts, plant organs, fruits, roots, leaves, flowers, seeds or other propagation material including tubers comprising a recombinant gene according to the invention, particularly a recombinant gene wherein the following DNA regions are operably linked:
a) a light-inducible plant-expressible promoter; including a light-inducible promoter preferentially or selectively expressed in green tissue and/or mesophyll.
a) a DNA region encoding a subunit of the mitochondrial glycine decarboxylase complex such as the mitochondrial H-protein as herein described; and
b) optionally, a 3′ end region involved in transcription termination and polyadenylation, preferably a 3′ end region functional in plant cells.

The invention also provides the recombinant genes herein described, whether as DNA molecules, RNA molecules, comprised within a vector or plasmid, comprised within host cells, including microbial host cells and the like.

The invention also relates to the use of an mitochondrial protein H encoding DNA fragment to increase the photosynthesis and/or photorespiration in a plant or to increase yield, growth or biomass in a plant.

Plants obtained using the methods of the invention, or plants or parts thereof comprising the recombinant genes according to the invention can be used as food or feed, or otherwise processed as conventional plants. Such plants can also be treated agronomically as conventional plants.

The obtained transformed plant can be used in a conventional breeding scheme to produce more transformed plants with the same characteristics or to introduce the chimeric gene according to the invention in other varieties of the same or related plant species, or in hybrid plants. Seeds obtained from the transformed plants contain the chimeric genes of the invention as a stable genomic insert and are also encompassed by the invention.

The plants and seeds according to the invention may be further treated with a chemical compound, such as a chemical compound selected from the following lists:

Herbicides: Clethodim, Clopyralid, Diclofop, Ethametsulfuron, Fluazifop, Glufosinate, Glyphosate, Metazachlor, Quinmerac, Quizalofop, Tepraloxydim, Trifluralin.

Fungicides/PGRs: Azoxystrobin, N-[9-(dichloromethylene)-1,2,3,4-tetrahydro-1,4-methanonaphthalen-5-yl]-3-(difluoromethyl)-1-methyl-1H-pyrazole-4-carboxamide (Benzovindiflupyr, Benzodiflupyr), Bixafen, Boscalid, Carbendazim, Carboxin, Chlormequat-chloride, Coniothryrium minitans, Cyproconazole, Cyprodinil, Difenoconazole, Dimethomorph, Dimoxystrobin, Epoxiconazole, Famoxadone, Fluazinam, Fludioxonil, Fluopicolide, Fluopyram, Fluoxastrobin, Fluquinconazole, Flusilazole, Fluthianil, Flutriafol, Fluxapyroxad, Iprodione, Isopyrazam, Mefenoxam, Mepiquat-chloride, Metalaxyl, Metconazole, Metominostrobin, Paclobutrazole, Penflufen, Penthiopyrad, Picoxystrobin, Prochloraz, Prothioconazole, Pyraclostrobin, Sedaxane, Tebuconazole, Tetraconazole, Thiophanate-methyl, Thiram, Triadimenol, Trifloxystrobin, Bacillus firmus, Bacillus firmus strain 1-1582, Bacillus subtilis, Bacillus subtilis strain GB03, Bacillus subtilis strain QST 713, Bacillus pumulis, Bacillus. pumulis strain GB34.

Insecticides: Acetamiprid, Aldicarb, Azadirachtin, Carbofuran, Chlorantraniliprole (Rynaxypyr), Clothianidin, Cyantraniliprole (Cyazypyr), (beta-)Cyfluthrin, gamma-Cyhalothrin, lambda-Cyhalothrin, Cypermethrin, Deltamethrin, Dimethoate, Dinetofuran, Ethiprole, Flonicamid, Flubendiamide, Fluensulfone, Fluopyram, Flupyradifurone, tau-Fluvalinate, Imicyafos, Imidacloprid, Metaflumizone, Methiocarb, Pymetrozine, Pyrifluquinazon, Spinetoram, Spinosad, Spirotetramate, Sulfoxaflor, Thiacloprid, Thiamethoxam, 1-(3-chloropyridin-2-yl)-N-[4-cyano-2-methyl-6-(methylcarbamoyl)phenyl]-3-{[5-(trifluoromethyl)-2H-tetrazol-2-yl]methyl}-1H-pyrazole-5-carboxamide, 1-(3-chloropyridin-2-yl)-N-[4-cyano-2-methyl-6-(methylcarbamoyl)phenyl]-3-{[5-(trifluoromethyl)-1H-tetrazol-1-yl]methyl}-1H-pyrazole-5-carboxamide, 1-{2-fluoro-4-methyl-5-[(2,2,2-trifluorethyl)sulfinyl]phenyl}-3-(trifluoromethyl)-1H-1,2,4-triazol-5-amine, (1E)-N-[(6-chloropyridin-3-yl)methyl]-N′-cyano-N-(2,2-difluoroethyl)ethanimidamide, Bacillus firmus, Bacillus firmus strain 1-1582, Bacillus subtilis, Bacillus subtilis strain GB03, Bacillus subtilis strain QST 713, Metarhizium anisopliae F52.

By “encoding” or “encoded,” with respect to a specified nucleic acid, is meant comprising the information for transcription into an RNA and in some embodiments, translation into the specified protein. A nucleic acid encoding a protein may comprise non-translated sequences (e.g., introns) within translated regions of the nucleic acid, or may lack such intervening non-translated sequences (e.g., as in cDNA). The information by which a protein is encoded is specified by the use of codons. Typically, the amino acid sequence is encoded by the nucleic acid using the “universal” genetic code.

As used herein “comprising” is to be interpreted as specifying the presence of the stated features, integers, steps or components as referred to, but does not preclude the presence or addition of one or more features, integers, steps or components, or groups thereof. Thus, e.g., a nucleic acid or protein comprising a sequence of nucleotides or amino acids, may comprise more nucleotides or amino acids than the actually cited ones, i.e., be embedded in a larger nucleic acid or protein. A chimeric gene comprising a DNA region, which is functionally or structurally defined, may comprise additional DNA regions etc.

The following non-limiting Examples describe the methods for increasing photorespiration, photosynthesis and increased growth. Unless stated otherwise in the Examples, all recombinant DNA techniques are carried out according to standard protocols as described in Sambrook et al. (1989) Molecular Cloning: A Laboratory Manual, Second Edition, Cold Spring Harbor Laboratory Press, NY and in Volumes 1 and 2 of Ausubel et al. (1994) Current Protocols in Molecular Biology, Current Protocols, USA. Standard materials and methods for plant molecular work are described in Plant Molecular Biology Labfax (1993) by R. D. D. Croy, jointly published by BIOS Scientific Publications Ltd (UK) and Blackwell Scientific Publications, UK.

Throughout the description and Examples, reference is made to the following sequences represented in the sequence listing:
SEQ ID No 1: amino acid sequence of the mitochondrial H-protein from Flaveria pringlei
SEQ ID No 2: nucleotide sequence of the mitochondrial H-protein from Flaveria pringlei
SEQ ID No. 3: nucleotide sequence of ST-LS1 promoter from Solanum tuberosum
SEQ ID No. 4: primer FpGLDH-SacI-S
SEQ ID No. 5: primer FpGLDH-EcoRI-AS
SEQ ID No. 6: primer ST-LS1-SacI-S
SEQ ID No. 7: primer ST-LS1-BamHI-AS

### EXAMPLES

### Example 1

### Materials and Methods

### 1.1. Overexpression Constructs, Transformation, and Plant Growth

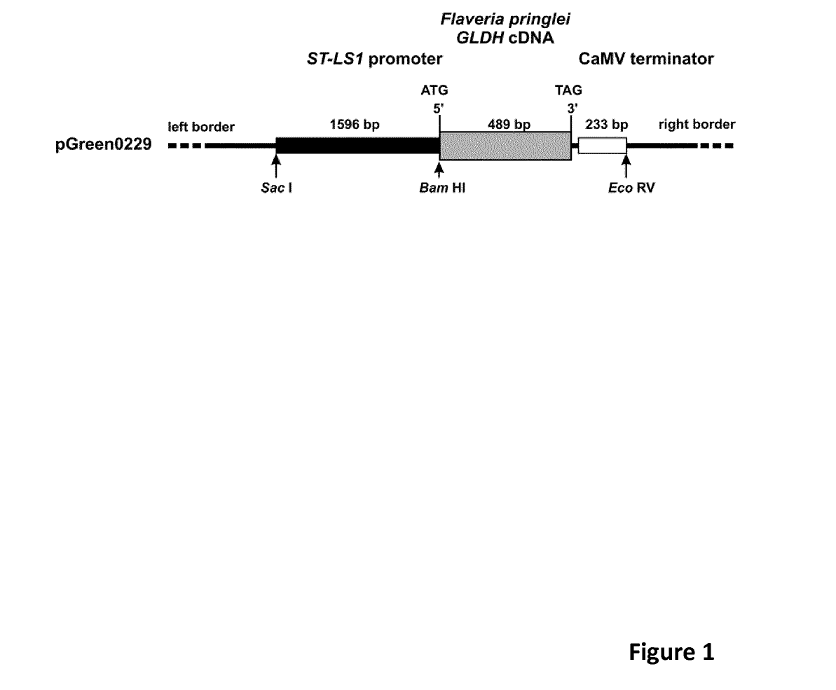

The entire coding sequence for the GDC H-protein (GLDH) was PCR-amplified from Flaveria pringlei cDNA HFP4 [25] using primers FpGLDH-SacI-S(5′-GAG CTC ATG GCT CTT AGA ATC TGG GCT-3; SEQ ID No: 4) and FpGLDH-EcoRI-AS (5′-GAA TTC CTA CGTG AGC AGA ATC TTC TTC-3′ SEQ ID No: 5). This amplificate was ligated into vector pGEMT (Invitrogen) and its correct sequence confirmed. The Sac I-Eco RI fragment was excised and ligated in front of the CaMV polyA site of the pGreen 35S-CaMV cassette (http://www.pgreen.ac.uk) to generate GLDH: CaMV. The ST-LS1 promoter sequence [26] was PCR-amplified from vector L700-pBIN19 [17] using primers ST-LS1-SacI-S(5′-GAG CTC GGC TTG ATT TGT TAG AAA ATT-3 SEQ ID No: 6) and ST-LS1-BamHI-AS (5′-GGA TCC TTT CTC CTA TAC CTT TTT TCT-3; SEQ ID No: 7), ligated into the binary plant transformation vector pGreen0229 [27] via the introduced Sac I and Bam HI sites, and complemented with the GDC-H:CaMV fragment via Bam HI and Eco RV sites. This construct (schematically shown in FIG. 1) was introduced into Agrobacterium tumefaciens strain GV3101 and used for the transformation [28] of Arabidopsis thaliana ecotype Col-0 (Arabidopsis). 22 phosphinotricine (Basta) resistant lines were isolated and preselected according to their leaf GDC-H content. Then, stable T3 lines were generated, and four of these lines displaying intermediate (lines FpH L16 and L17) and high H-protein overexpression (lines FpH L15 and L18) selected for further examination. For all analyses, we used plants grown at environmentally controlled conditions (10/14 h day/night-cycle, 20/18° C., ˜150 μmol·m⁻²·s⁻¹ photosynthetically active radiation) [29] to stadium 5.1 as defined in Boyes et al. [30].

### 1.2. Immunological Studies

SDS-PAGE of whole leaf protein extracts and protein gel blotting experiments were performed according to standard protocols using antibodies raised against recombinant H-protein (Flaveria trinervia), P-protein (Flaveria anomala), and T-protein (Solanum tuberosum).

### 1.3. Gas Exchange and Fluorescence Measurements

Gas exchange measurements were performed as previously described [29]. Night-respiration rates were determined 4 h after switching off the lights during the normal day/night cycle. Maximum PSII quantum yields (Fᵥ/Fₘ) and relative electron transport rates (ETR) at varying photosynthetic photon flux densities (PPFD) were measured using an Imaging PAM (M series, Walz, [31]). In short, basal fluorescence (F₀) was measured with dark-adapted leaves and steady-state fluorescence (Fₛ) with varying intensities of actinic light. Maximum fluorescence (dark-adapted leaves, Fₘ; illuminated leaves, Fₘ′) was induced with saturating white-light pulses (5,000 μmol m⁻² s⁻¹). Fᵥ/Fₘ was calculated as (Fₘ−F₀)/Fₘ and the effective quantum yield of PSII as YPSII=(Fₘ′−Fₛ)/Fₘ′ according to Genty [32]. From these values, absolute electron transport rates were calculated as ETR=YPSII·PPFD·0.84·0.5, assuming that 84% of the incident quanta are absorbed by the leaf and that linear electron transport requires two quanta per electron. The light saturation point (LSP) is the PPFD that causes 90% of the maximum ETR (ETRₘₐₓ).

### 1.4. Metabolite Profiling

Rosette leaf samples were harvested in the middle of the light period (after 5 h) and analysed as described elsewhere for the GC-MS-based method [33] and for the LC-MS-based method [34].

### 1.5. Statistical Analysis

Analysis of variance (ANOVA) was performed for all data using the Holm-Sidak test for comparisons (Sigma Plot 11, Systat Software Inc.).

### Example 2

### Overexpression of GDCH Results in Increased Growth

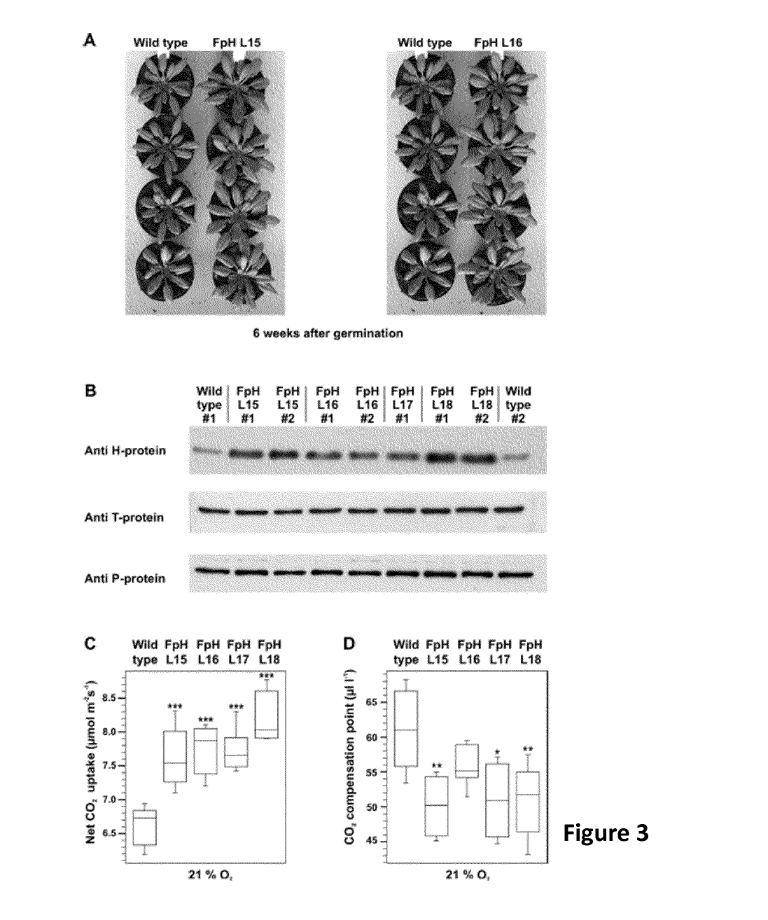

Since it is known that elevated H-protein concentrations increase P-protein activity in vitro [35], we chose this particular GDC component protein for overexpression in Arabidopsis. To avoid RNA interference and provide adequate transcriptional regulation, we fused cDNA encoding a Flaveria pringlei H-protein [25] to the leaf-specific and light-regulated Solanum tuberosum ST-LS1 promoter [26] and used this construct to stably transform wild-type Arabidopsis [28]. Transgenic lines were preselected from a total of 22 Basta-resistant lines according to their leaf H-protein content and selfed over several generations. Two T3-generation lines displaying intermediate (line FpH L17) and high (line FpH L18) H-protein overexpression were examined for photosynthetic-photorespiratory properties, metabolite contents, and growth. A less comprehensive data set obtained with two more overexpressor lines (FpH L15 and L16) is shown in FIG. 3. To exclude seed-age related bias, wild-type seed of the same harvest was used for growing the control plants.

In comparison to simultaneously (randomized side-by-side) grown wild-type Arabidopsis, a distinct growth promotion of the overexpressor lines became apparent already several weeks after germination and was fully established after six weeks (FIG. 1: Schematic representation of the overexpression construct harboring cDNA encoding Flaveria pringlei H-protein [25] under control of the Solanum tuberosum ST-LS1 promoter [26].

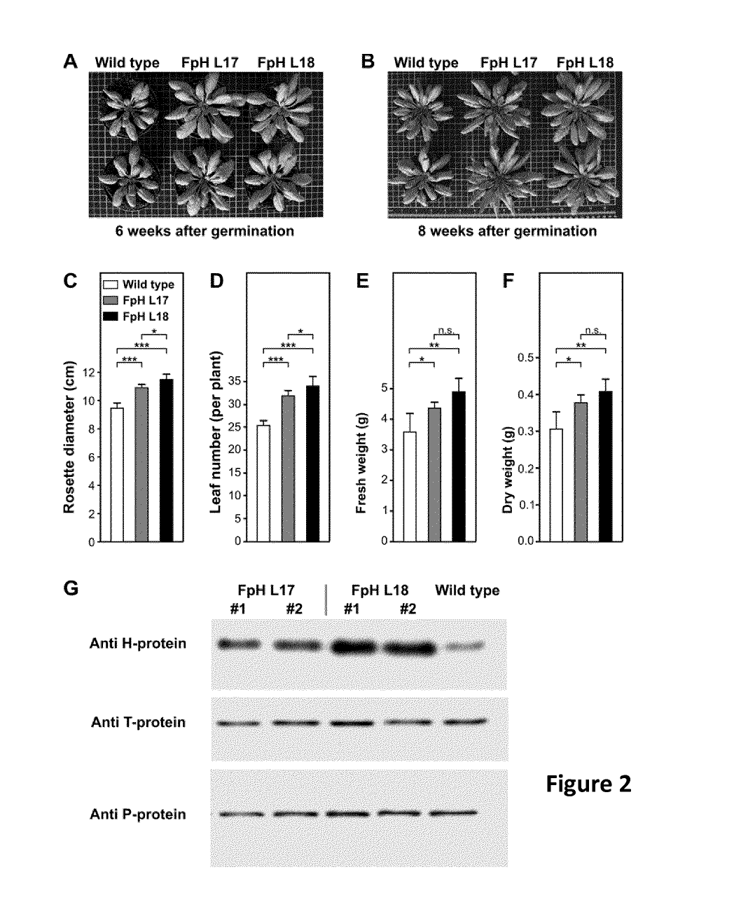

FIGS. 2 A and B). In quantitative terms, the overexpressor lines displayed significantly larger rosettes (FIG. 2 C) and more leaves per plant (FIG. 2D) in combination with significantly longer (wild type, 3.75±0.13; FpH L17, 3.98±0.08; FpH L18, 4.34±0.16 cm) and broader (wild type, 1.60±0.15; FpH L17, 1.89±0.10; FpH L18, 1.95±0.07 cm) rosette leaves. These improved growth features summed up to 37% higher fresh (FIG. 2E) and 33% higher dry weight (FIG. 2 F) in the best-performing line FpH L18. These growth features correlated nicely with about 2.5-fold (FpH L17) or five-fold (FpH L18) elevated leaf H-protein levels (FIG. 2 G). Total leaf contents of P- and T-protein remained unaltered (FIG. 2 G). Germination and the time until flowering were also unaltered relative to wild-type plants.

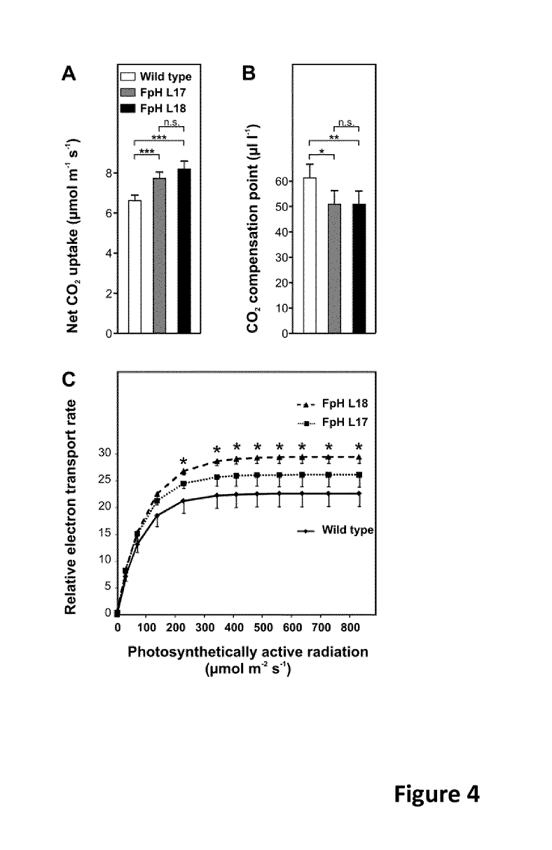

The improved growth of the H-protein overexpressor lines was associated with significantly accelerated net-CO₂ uptake rates (FIG. 4A and FIG. 3). Moreover, we observed significantly lower CO₂ compensation points (F) in three out of four examined overexpressor lines (FIG. 4 B and FIG. 3). In land plants of the C3 photosynthetic type, which include Arabidopsis, F is a very sensitive indicator of the balance between photosynthetic CO₂ uptake and (photo)respiratory CO₂ release. Our data suggest that this balance is affected by the catalytic capacity of the GDC reaction that, on its part, depends on the amount of available H-protein.

At a five-fold elevated CO₂ concentration, which considerably suppresses photorespiration, statistically significant differences in net-CO₂ uptake between the wild type and overexpressor lines could not be discerned any more (wild type, 13.39±0.42; FpH L17, 13.83±0.41; FpH L18, 13.94±0.75). This further supports our notion that the enhanced photosynthetic CO₂ uptake is the result of an alleviated photorespiratory carbon flow, brought about by higher GDC activity. Plant growth, to a large extent, occurs during the night and is driven by the use of accumulated stocks of transitional starch for respiration [36]. Hence, though we did not measure starch contents, the 20% (in FpH L17) and 24% (in FpH L18) enhanced rates of night respiration (wild type, 0.70±0.07; FpH L17, 0.84±0.17; FpH L18, 0.87±0.09) fit nicely to the better plant growth demonstrated in FIGS. 2 A and 2 B.

In order to examine whether these alterations in photosynthetic gas exchange affect the photosynthetic electron transport, we measured maximum PSII quantum yields (Fᵥ/Fₘ) and relative electron transport rates (ETR) at varying light intensities (Table1; FIG. 4C). Fᵥ/Fₘ values were very similar in the wild type and the overexpressor lines, but both the ETR values and the light saturation points were significantly higher in the plants containing more GDC H-protein, especially at high light intensities. This observation indicates that the improvements to the photosynthetic-photorespiratory carbon flow in turn cause an accelerated electron flow at PSII.

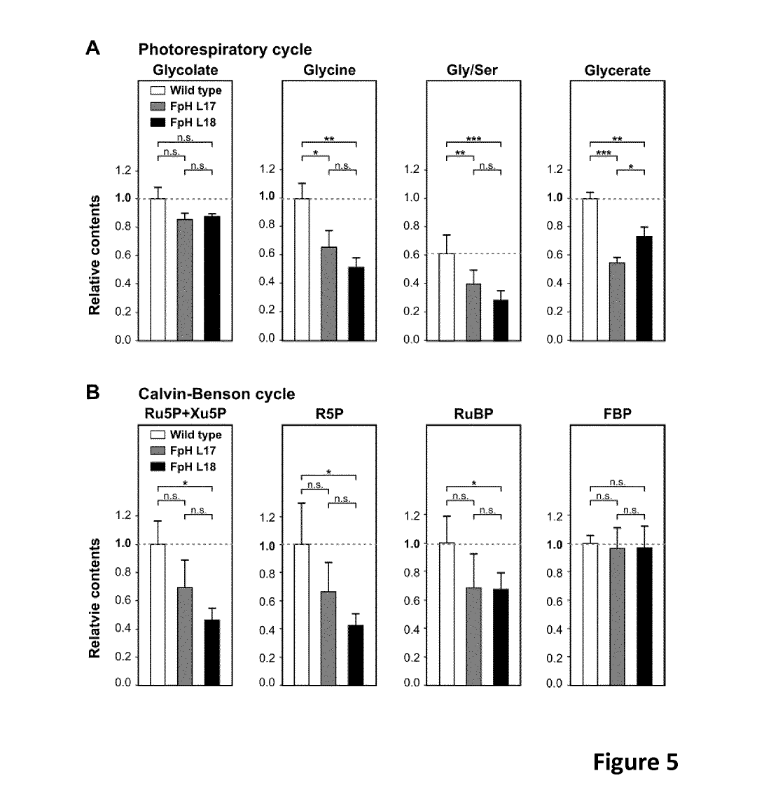

An alleviation of a restriction in photorespiratory carbon flow should ultimately result in lower steady-state concentrations of photorespiratory metabolites. In the case of elevated GDC activity, one would anticipate reduced glycine levels. Indeed, metabolite profiling by GC-MS revealed an up to a significant 34-48% reduction of the leaf glycine content and the glycine-to-serine ratio in both overexpressor lines (FIG. 5A). Except some changes in the levels of several other photorespiratory metabolites upstream (non-significant 12-15% decrease of glycolate) and downstream (20-60% increase in hydroxypyruvate, significant 27-45% decrease of glycerate) of the GDC reaction, the levels of most other metabolites remained unaltered (Table 2). The only other metabolites showing a significant change in both lines were asparagine and fructose.

Once the inhibition by photorespiratory metabolites is partially relieved the CO₂-fixing part of the CB cycle should become a stronger sink for RuBP. This is what we observed as well: changes to CB cycle metabolites were minor to nil—except considerably lower values for RuBP, pentulose 5-phosphates, and ribose 5-phosphate in the H-protein overexpressing lines (FIG. 3B and Table 3). Again, this effect was approximately correlated with the amount of extra H-protein. The slight increase of fructose 6-phosphate, glucose 6-phosphate, and glucose 1-phosphate, which are intermediates in the pathway of sucrose synthesis, is consistent with the higher rates of photosynthesis.

Summarizing, our findings demonstrate regulatory interaction between the photorespiratory pathway and the CB cycle. A plausible explanation could be that some photorespiratory metabolites, for example glyoxylate or glycine, exert negative feed-back to down-regulate CB cycle enzymes. Our experiments suggest that this feed-back inhibition can be artificially relaxed by decreasing the accumulation of intermediates of the photorespiratory pathway, in particular at the glycine-to-serine conversion step. This effect is best visible by the reduced leaf content of glycine in combination with accelerated CO₂ fixation and a consequently lower RuBP level. From an ecophysiological point of view, the observed interaction might represent a useful strategy of C₃ plants to simultaneously down-regulate photosynthesis and photorespiration at high-photorespiration conditions, for example at high temperatures or suboptimum water supply. The operation and fine-tuning of this regulation remain to be investigated. As far as the photorespiratory side is concerned, GDC appears as one of the key signallers in this network.

### Tables

TABLE 1
PSII fluorescence parameters and relative photosynthetic electron
transport. Data for maximum quantum efficiency of PSII (Fv/Fm),
electron transport rate efficiency at low light intensity (alpha),
maximum relative electron transport rate (ETRmax), and the
light saturation point (LSP) are shown as mean values ± SD
from at least five individual plants (5 areas of interest each per plant).
Asterisks indicate significant differences relative to side-by-side
grown wild-type plants (*p < 0.05).

| | Wild type | FpH L17 | FpH L18 |
|---|---|---|---|
| Fᵥ/Fₘ | 0.7572 ± 0.0172 | 0.7587 ± 0.0097 | 0.7662 ± 0.0041 |
| alpha | 0.3107 ± 0.0406 | 0.3263 ± 0.0615 | 0.3089 ± 0.0431 |
| ETRₘₐₓ | 23.12 ± 2.94 | 26.18 ± 2.48 | 29.49 ± 1.37* |
| LSP | 171.68 ± 13.12 | 180.03 ± 28.58 | 202.97 ± 13.21* |

TABLE 2
Leaf metabolite profiling of H-protein overexpressors (GC-MS). Samples
were taken at mid-day (5 h light) and analyzed by GC-MS [33]. Shown
are mean values ± SD for leaf samples from at least five individual
plants. Asterisks indicate significant differences relative to side-by-side
grown wild-type plants (*p < 0.05; **p < 0.01). Values in bold
were used for FIG. 3A.
Changes in leaf metabolite contents (relative to the wild type)

| | Wild type | FpH L17 | FpH L18 |
|---|---|---|---|
| Alanine | 1.00 ± 0.11 | 1.60 ± 0.08** | 1.20 ± 0.06 |
| β-Alanine | 1.00 ± 0.10 | 1.14 ± 0.07 | 1.21 ± 0.11 |
| Arginine | 1.00 ± 0.16 | 0.92 ± 0.04 | 0.92 ± 0.12 |
| Ascorbate | 1.00 ± 0.17 | 1.05 ± 0.23 | 1.40 ± 0.55 |
| Asparagine | 1.00 ± 0.05 | 0.82 ± 0.04* | 0.73 ± 0.10* |
| Aspartate | 1.00 ± 0.09 | 0.80 ± 0.06 | 0.84 ± 0.09 |
| Citrate | 1.00 ± 0.04 | 1.01 ± 0.11 | 0.97 ± 0.08 |
| Dehydroascorbate | 1.00 ± 0.07 | 0.97 ± 0.06 | 1.03 ± 0.08 |
| Erythritol | 1.00 ± 0.06 | 1.00 ± 0.11 | 1.21 ± 0.06* |
| Ethanolamine | 1.00 ± 0.05 | 0.93 ± 0.06 | 0.99 ± 0.07 |
| Fructose | 1.00 ± 0.13 | 0.50 ± 0.14* | 0.45 ± 0.14* |
| Fumarate | 1.00 ± 0.04 | 0.96 ± 0.06 | 1.08 ± 0.07 |
| GABA | 1.00 ± 0.09 | 0.72 ± 0.05* | 1.06 ± 0.09 |
| Galactinol | 1.00 ± 0.22 | 1.15 ± 0.28 | 0.82 ± 0.12 |
| Galactose | 1.00 ± 0.18 | 1.45 ± 0.41 | 0.91 ± 0.05 |
| Glucose | 1.00 ± 0.05 | 0.89 ± 0.16 | 1.01 ± 0.11 |
| Glutamate | 1.00 ± 0.21 | 1.49 ± 0.35 | 1.05 ± 0.15 |
| Glutamine | 1.00 ± 0.20 | 0.89 ± 0.09 | 0.68 ± 0.07 |
| Glycerate | 1.00 ± 0.04 | 0.55 ± 0.04** | 0.73 ± 0.06* |
| Glycerol | 1.00 ± 0.09 | 0.80 ± 0.04 | 0.81 ± 0.03 |
| Glycine | 1.00 ± 0.11 | 0.66 ± 0.12* | 0.52 ± 0.06** |
| Glycolate | 1.00 ± 0.08 | 0.85 ± 0.04 | 0.88 ± 0.02 |
| Guanidine | 1.00 ± 0.25 | 0.83 ± 0.10 | 1.03 ± 0.24 |
| Hydroxybutyrate | 1.00 ± 0.08 | 0.71 ± 0.05 | 0.75 ± 0.16 |
| Hydroxypyruvate | 1.00 ± 0.11 | 1.60 ± 0.09 | 1.20 ± 0.06 |
| Inositol | 1.00 ± 0.04 | 0.95 ± 0.05 | 1.05 ± 0.06 |
| Isoleucine | 1.00 ± 0.09 | 0.89 ± 0.02 | 0.88 ± 0.05 |
| α-Ketoglutarate | 1.00 ± 0.10 | 1.22 ± 0.08 | 1.24 ± 0.10 |
| Lysine | 1.00 ± 0.08 | 0.90 ± 0.06 | 0.87 ± 0.07 |
| Malate | 1.00 ± 0.03 | 0.96 ± 0.05 | 1.03 ± 0.06 |
| Maltose | 1.00 ± 0.20 | 0.60 ± 0.06 | 0.68 ± 0.08 |
| Mannose | 1.00 ± 0.14 | 1.00 ± 0.22 | 0.85 ± 0.09 |
| Methionine | 1.00 ± 0.06 | 0.79 ± 0.10 | 0.97 ± 0.05 |
| Nicotinic acid | 1.00 ± 0.07 | 0.98 ± 0.09 | 1.13 ± 0.09 |
| Ornithine | 1.00 ± 0.08 | 0.86 ± 0.07 | 0.64 ± 0.09* |
| Phenylalanine | 1.00 ± 0.07 | 0.84 ± 0.06 | 0.88 ± 0.09 |
| Phosphoric acid | 1.00 ± 0.16 | 0.68 ± 0.15 | 0.86 ± 0.05 |
| Proline | 1.00 ± 0.27 | 0.33 ± 0.16 | 0.19 ± 0.06* |
| Putrescine | 1.00 ± 0.14 | 0.88 ± 0.11 | 1.24 ± 0.10 |
| Pyruvate | 1.00 ± 0.16 | 0.77 ± 0.06 | 0.82 ± 0.07 |
| Raffinose | 1.00 ± 0.31 | 0.93 ± 0.27 | 0.74 ± 0.05 |
| Rhamnose | 1.00 ± 0.09 | 1.07 ± 0.08 | 1.02 ± 0.11 |
| Ribose | 1.00 ± 0.07 | 0.92 ± 0.08 | 1.09 ± 0.03 |
| Serine | 1.00 ± 0.09 | 1.04 ± 0.07 | 0.96 ± 0.05 |
| Shikimate | 1.00 ± 0.14 | 0.91 ± 0.05 | 0.92 ± 0.09 |
| Sorbose | 1.00 ± 0.18 | 0.61 ± 0.19 | 0.57 ± 0.18 |
| Spermidine | 1.00 ± 0.09 | 1.01 ± 0.19 | 0.57 ± 0.18 |
| Succinate | 1.00 ± 0.17 | 0.73 ± 0.08 | 0.66 ± 0.14 |
| Sucrose | 1.00 ± 0.04 | 0.96 ± 0.05 | 1.06 ± 0.06 |
| Threonic acid | 1.00 ± 0.09 | 1.14 ± 0.12 | 1.10 ± 0.07 |
| Threonine | 1.00 ± 0.09 | 0.93 ± 0.04 | 0.93 ± 0.06 |
| Trehalose | 1.00 ± 0.06 | 0.66 ± 0.15 | 0.70 ± 0.14 |
| Tryptophan | 1.00 ± 0.01 | 1.12 ± 0.08 | 1.02 ± 0.10 |
| Tyrosine | 1.00 ± 0.06 | 0.94 ± 0.02 | 0.95 ± 0.08 |
| Valine | 1.00 ± 0.09 | 0.87 ± 0.04 | 0.81 ± 0.04 |

TABLE 3
Leaf metabolite profiling of H-protein overexpressors (LC-MS). Samples
were taken at mid-day (5 h light) and analyzed by LC-MS [34]. Shown are (A)
mean absolute and (B) relative-to-wild-type values ± SD for leaf samples from four
individual plants. Asterisks indicate significant differences relative to side-by-side
grown wild-type plants (*p < 0.05; **p < 0.01). Relative values in bold in Table
B were used for FIG. 3B.

| | | |
|---|---|---|
| Wild type | FpH L17 | FpH L18 |

A. Leaf metabolite contents (nmol g−1 fresh weight)

| | | | |
|---|---|---|---|
| ADP | 16.8 ± 1.9 | 15.5 ± 2.1 | 18.9 ± 0.9 |
| ADP-glucose | 1.0 ± 0.3 | 1.1 ± 0.2 | 1.1 ± 0.2 |
| AMP | 29.0 ± 7.5 | 20.0 ± 1.4 | 19.5 ± 2.0 |
| Aspartate | 1520.8 ± 283.9 | 1232.6 ± 214.3 | 1322.1 ± 42.9 |
| Dihydroxyacetone-P | 13.2 ± 2.9 | 10.7 ± 1.1 | 10.4 ± 2.3 |
| Fructose 6-P | 71.5 ± 12.5 | 83.8 ± 6.5 | 89.7 ± 6.2* |
| Fructose 1,6-bP | 3.1 ± 0.2 | 3.0 ± 0.5 | 3.0 ± 0.5 |
| Glucose 1-P | 17.9 ± 1.6 | 19.7 ± 1.3 | 21.7 ± 3.0 |
| Glucose 6-P | 159.2 ± 17.4 | 173.1 ± 11.0 | 170.6 ± 24.6 |
| Glutamate | 2733.7 ± 121.1 | 2451.7 ± 455.8 | 2246.0 ± 236.8** |
| Glycerate | 522.3 ± 101.0 | 466.2 ± 56.0 | 305.3 ± 70.4* |
| α-Ketoglutarate | 132.6 ± 32.8 | 141.8 ± 21.7 | 107.5 ± 15.0 |
| Malate | 11147 ± 1217 | 10580 ± 847 | 9429 ± 2300 |
| NAD | 14.7 ± 1.1 | 17.4 ± 1.0* | 14.7 ± 1.0 |
| NADP | 8.1 ± 0.8 | 7.3 ± 1.0 | 7.1 ± 1.6 |
| Ribose 5-P | 6.2 ± 1.8 | 4.1 ± 1.3 | 2.6 ± 0.5** |
| Ribulose 5-P/Xylulose 5-P | 34.7 ± 5.7 | 24.0 ± 6.8 | 16.1 ± 2.8** |
| Ribulose 1,5-bP | 42.0 ± 8.2 | 28.8 ± 10.3 | 28.3 ± 5.1* |
| Seduheptulose 7-P | 33.8 ± 11.6 | 40.3 ± 2.7 | 46.1 ± 6.7 |
| Seduheptulose 1,7-bP | 1.8 ± 0.3 | 1.5 ± 0.2 | 1.6 ± 0.8 |
| Shikimate | 19.7 ± 5.2 | 26.3 ± 4.5 | 23.0 ± 3.4 |
| UDP-glucose | 86.0 ± 4.8 | 87.1 ± 6.5 | 89.8 ± 14.5 |

B. Changes in leaf metabolite contents (relative to the wild type)

| | | | |
|---|---|---|---|
| ADP | 1.00 ± 0.11 | 0.93 ± 0.13 | 1.13 ± 0.05 |
| ADP-glucose | 1.00 ± 0.26 | 1.15 ± 0.18 | 1.18 ± 0.18 |
| AMP | 1.00 ± 0.26 | 0.69 ± 0.05 | 0.67 ± 0.07 |
| Aspartate | 1.00 ± 0.19 | 0.81 ± 0.14 | 0.87 ± 0.03 |
| Dihydroxyacetone-P | 1.00 ± 0.22 | 0.81 ± 0.09 | 0.79 ± 0.18 |
| Fructose 6-P | 1.00 ± 0.17 | 1.17 ± 0.09 | 1.25 ± 0.09* |
| Fructose 1,6-bP | 1.00 ± 0.06 | 0.96 ± 0.15 | 0.97 ± 0.16 |
| Glucose 1-P | 1.00 ± 0.09 | 1.10 ± 0.07 | 1.25 ± 0.09 |
| Glucose 6-P | 1.00 ± 0.11 | 1.09 ± 0.07 | 1.07 ± 0.15 |
| Glutamate | 1.00 ± 0.04 | 0.90 ± 0.17 | 0.82 ± 0.07** |
| Glycerate | 1.00 ± 0.18 | 0.84 ± 0.10 | 0.55 ± 0.13* |
| α-Ketoglutarate | 1.00 ± 0.25 | 1.07 ± 0.16 | 0.81 ± 0.11 |
| Malate | 1.00 ± 0.11 | 0.94 ± 0.08 | 0.84 ± 0.20 |
| NAD | 1.00 ± 0.08 | 1.18 ± 0.07* | 1.00 ± 0.07 |
| NADP | 1.00 ± 0.09 | 0.91 ± 0.13 | 0.88 ± 0.20 |
| Ribose 5-P | 1.00 ± 0.30 | 0.67 ± 0.20 | 0.42 ± 0.09** |
| Ribulose 5-P/Xylulose 5-P | 1.00 ± 0.17 | 0.69 ± 0.20 | 0.46 ± 0.08** |
| Ribulose 1,5.bP | 1.00 ± 0.20 | 0.69 ± 0.24 | 0.67 ± 0.12* |
| Seduheptulose 7-P | 1.00 ± 0.34 | 1.19 ± 0.08 | 1.36 ± 0.20 |
| Seduheptulose 1,7-bP | 1.00 ± 0.14 | 0.83 ± 0.09 | 0.90 ± 0.44 |
| Shikimate | 1.00 ± 0.27 | 1.34 ± 0.23 | 1.17 ± 0.17 |
| UDP-glucose | 1.00 ± 0.06 | 1.01 ± 0.08 | 1.04 ± 0.17 |

### REFERENCES NOT INDICATED IN THE TEXT

[1] Cleland W. W., Andrews T. J., Gutteridge S., Hartman F. C. and Lorimer G. H. (1998) Mechanism of Rubisco: the carbamate as general base. Chem. Rev. 98, 549-562.
[2] Galmés J., Flexas J., Cifre J., Medrano H., Keys A. J., Mitchell R. A. C., Madgwick P. J., Haslam R. P. and Parry M. A. J. (2005) Rubisco specificity factor tends to be larger in plant species from drier habitats and in species with persistent leaves. Plant Cell Environ. 28, 571-579.
[3] Anderson L. E. (1971) Chloroplast and cytoplasmic enzymes. 2. Pea leaf triose phosphate isomerases. Biochim. Biophys. Acta 235, 237-244.
[4] Kelly G. J. and Latzko E. (1976) Inhibition of spinach-leaf phosphofructokinase by 2-phosphoglycollate. FEBS Lett. 68, 55-58.
[5] Foyer C. H., Bloom A. J., Queval G. and Noctor G. (2009) Photorespiratory metabolism: genes, mutants, energetics, and redox signaling. Annu. Rev. Plant Biol. 60, 455-484.
[6] Tolbert N. E. (1997) The C2 oxidative photosynthetic carbon cycle. Annu. Rev. Plant Physiol. Plant Mol. Biol. 48, 1-25.
[7] Bauwe H., Hagemann M. and Fernie A. R. (2010) Photorespiration: players, partners and origin. Trends Plant Sci. 15, 330-336.
[8] Whitney S. M., Houtz R. L. and Alonso H. (2011) Advancing our understanding and capacity to engineer nature's CO₂-sequestering enzyme, Rubisco. Plant Physiol. 155, 27-35.
[9] Maurino V. G. and Peterhansel C. (2010) Photorespiration: current status and approaches for metabolic engineering. Curr. Opin. Plant Biol. 13, 248-255.
[10] von Caemmerer S. and Evans J. R. (2010) Enhancing C₃ photosynthesis. Plant Physiol. 154, 589-592.
[11] Raines C. A. (2011) Increasing photosynthetic carbon assimilation in C₃ plants to improve crop yield: current and future strategies. Plant Physiol. 155, 36-42.
[12] Hibberd J. M., Sheehy J. E. and Langdale J. A. (2008) Using C₄ photosynthesis to increase the yield of rice—rationale and feasibility. Curr. Opin. Plant Biol. 11, 228-231.
[13] Eisenhut M., Ruth W., Haimovich M., Bauwe H., Kaplan A. and Hagemann M. (2008) The photorespiratory glycolate metabolism is essential for cyanobacteria and might have been conveyed endosymbiontically to plants. Proc. Natl. Acad. Sci. USA 105, 17199-17204.
[14] Hackenberg C., Kern R., Hüge J., Stal L. J., Tsuji Y., Kopka J., Shiraiwa Y., Bauwe H. and Hagemann M. (2011) Cyanobacterial lactate oxidases serve as essential partners in N₂ fixation and evolved into photorespiratory glycolate oxidases in plants. Plant Cell 23, 2978-2990.
[15] Zelitch I., Schultes N. P., Peterson R. B., Brown P. and Brutnell T. P. (2009) High glycolate oxidase activity is required for survival of maize in normal air. Plant Physiol. 149, 195-204.
[16] Servaites J. C. and Ogren W. L. (1977) Chemical inhibition of the glycolate pathway in soybean leaf cells. Plant Physiol. 60, 461-466.
[17] Heineke D., Bykova N., Gardeström P. and Bauwe H. (2001) Metabolic response of potato plants to an antisense reduction of the P-protein of glycine decarboxylase. Planta 212, 880-887.
[18] Wingler A., Lea P. J. and Leegood R. C. (1997) Control of photosynthesis in barley plants with reduced activities of glycine decarboxylase. Planta 202, 171-178.
[19] Chastain C. J. and Ogren W. L. (1989) Glyoxylate inhibition of ribulosebisphosphate carboxylase/oxygenase activation state in vivo. Plant Cell Physiol. 30, 937-944.
[20] Campbell W. J. and Ogren W. L. (1990) Glyoxylate inhibition of ribulosebisphosphate carboxylase/oxygenase activation in intact, lysed, and reconstituted chloroplasts. Photosynth. Res. 23, 257-268.
[21] Häusler R. E., Bailey K. J., Lea P. J. and Leegood R. C. (1996) Control of photosynthesis in barley mutants with reduced activities of glutamine synthetase and glutamate synthase. 3. Aspects of glyoxylate metabolism and effects of glyoxylate on the activation state of ribulose-1,5-bisphosphate carboxylase-oxygenase. Planta 200, 388-396.
[22] Eisenhut M., Bauwe H. and Hagemann M. (2007) Glycine accumulation is toxic for the cyanobacterium Synechocystis sp. strain PCC 6803, but can be compensated by supplementation with magnesium ions. FEMS Microbiol. Lett. 277, 232-237.
[23] Kisaki T. and Tolbert N. E. (1970) Glycine as substrate for photorespiration. Plant Cell Physiol. 11, 247-258.
[24] Blackwell R. D., Murray A. J. S., Lea P. J., Kendall A., Hall N. P., Turner J. C. and Wallsgrove R. M. (1988) The value of mutants unable to carry out photorespiration. Photosynth. Res. 16, 155-176.
[25] Kopriva S. and Bauwe H. (1995) H-protein of glycine decarboxylase is encoded by multigene families in Flaveria pringlei and F. cronquistii (Asteraceae). Mol. Gen. Genet. 248, 111-116.
[26] Stockhaus J., Schell J. and Willmitzer L. (1989) Correlation of the expression of the nuclear photosynthetic gene ST-LS1 with the presence of chloroplast. EMBO J. 8, 2445-2451.
[27] Hellens R. P., Edwards E. A., Leyland N. R., Bean S. and Mullineaux P. M. (2000) pGreen: a versatile and flexible binary Ti vector for Agrobacterium-mediated plant transformation. Plant Mol. Biol. 42, 819-832.
[28] Clough S. J. and Bent A. F. (1998) Floral dip: a simplified method for Agrobacterium-mediated transformation of Arabidopsis thaliana. Plant J. 16, 735-743.
[29] Timm S., Florian A., Jahnke K., Nunes-Nesi A., Fernie A. R. and Bauwe H. (2011) The hydroxypyruvate-reducing system in Arabidopsis: Multiple enzymes for the same end. Plant Physiol. 155, 694-705.
[30] Boyes D. C., Zayed A. M., Ascenzi R., McCaskill A. J., Hoffman N. E., Davis K. R. and Gorlach J. (2001) Growth stage-based phenotypic analysis of Arabidopsis: A model for high throughput functional genomics in plants. Plant Cell 13, 1499-1510.
[31] Schreiber U., Schliwa U. and Bilger W. (1986) Continuous recording of photochemical and nonphotochemical chlorophyll fluorescence quenching with a new type of modulation fluorometer. Photosynth. Res. 10, 51-62.
[32] Genty B., Briantais J. M. and Baker N. R. (1989) The relationship between the quantum yield of photosynthetic electron transport and quenching of chlorophyll fluorescence. Biochim. Biophys Acta 990, 87-92.
[33] Lisec J., Schauer N., Kopka J., Willmitzer L. and Femie A. R. (2006) Gas chromatography mass spectrometry-based metabolite profiling in plants. Nat. Protoc. 1, 387-396.
[34] Arrivault S., Guenther M., Ivakov A., Feil R., Vosloh D., van Dongen J. T., Sulpice

R. and Stitt M. (2009) Use of reverse-phase liquid chromatography, linked to tandem mass spectrometry, to profile the Calvin cycle and other metabolic intermediates in Arabidopsis rosettes at different carbon dioxide concentrations. Plant J. 59, 826-839.
[35] Hasse D., Mikkat S., Hagemann M. and Bauwe H. (2009) Alternative splicing produces an H-protein with better substrate properties for the P-protein of glycine decarboxylase. FEBS J. 276, 6985-6991.
[36] Weise S. E., Weber A. P. and Sharkey T. D. (2004) Maltose is the major form of carbon exported from the chloroplast at night. Planta 218, 474-482.

1. A plant comprising a recombinant gene, said recombinant gene comprising the following operably linked DNA regions:
a. a light-inducible plant-expressible promoter;
b. a DNA region encoding a subunit of the mitochondrial glycine decarboxylase complex; and
c. optionally, a 3′ end region involved in transcription termination and polyadenylation, preferably a 3′ end region functional in plant cells.

2. The plant of claim 1, wherein said subunit of the glycine decarboxylase complex is the H-protein (glycine cleavage complex lipoylprotein).

3. The plant of claim 2, wherein said H-protein is an H-protein derived from a plant such as a seed-bearing plant including Aegilops tauschii, Arabidopsis lyrata, Arabidopsis thaliana, Beta vulgaris, Brachypodium distachyon, Cicer arietinum, Cucumis sativus, Flaveria anomala, Flaveria bidentis, Flaveria brownii, Flaveria chlorifolia, Flaveria cronquistii, Flaveria floridana, Flaveria linearis, Flaveria palmeri, Flaveria pringlei, Flaveria pubescens, Flaveria trinervia, Glycine max, Hordeum vulgare subsp. vulgare, Lotus japonica, Medicago truncatula, Oryza sativa Indica Group, Oryza sativa Japonica Group, Pinus pinaster, Pisum abyssinicum, Pisum fulvum, Pisum sativum subsp. elatius, Pisum sativum subsp. transcaucasicum, Pisum sativum var. pumilio, Pisum sativum var. tibetanicum, Pisum sativum, Populus tremuloides, Populus trichocarpa, Ricinus communis, Sonneratia alba, Sorghum bicolor, Sorghum bicolor, Triticum aestivum, Triticum urartu, Vitis vinifera or Zea mays.

4. The plant of claim 2, wherein said H-protein is an H-protein derived from an algal species including Micromonas or Chlamydomonas.

5. The plant of claim 2, wherein said H-protein comprises an amino acid sequence having at least 80% sequence identity with the amino acid sequence of SEQ ID NO. 1.

6. The plant of claim 1, wherein said subunit of the glycine decarboxylase complex is selected from the P-protein, the T-protein or the L-protein.

7. The plant of claim 1, wherein said light-inducible promoter is selected from a promoter of an LS1 gene, a promoter of Rubisco small subunit gene, or a promoter of a chlorophyll a/b binding protein gene.

8. The plant of claim 7, wherein said light-inducible promoter comprises the nucleotide sequence of SEQ ID NO. 3 from nucleotide 1 to nucleotide 1571.

9. The plant of claim 1, wherein said recombinant gene comprises a ST-LS1 promoter from Solanum tuberosum operably linked to a H-protein encoding region from Flaveria pringlei.

10. A plant with increased photosynthesis and/or photorespiration wherein the level of active H-protein in the mitochondria has been increased compared to a wild-type plant.

11. The plant of claim 10, wherein said level of active H-protein has been increased through using a recombinant gene expressing said H-protein under control of a heterologous promoter.

12. The plant of claim 11, wherein said heterologous promoter is a light-inducible, mesophyll-selective promoter.

13. The plant of claim 2, wherein said plant is selected from oilseed rape, cotton, rice, soybean, wheat, sugarcane or corn.

14. A recombinant gene as described in claim 1.

15. A method for increasing photosynthesis and/or photorespiration in a cell of a plant, a plant, or part of a plant comprising the step of providing a recombinant gene to cells of said plant, said recombinant gene comprising the following operably linked DNA fragments
a. a plant-expressible promoter;
b. a DNA region encoding a subunit of the mitochondrial glycine decarboxylase complex; and
c. optionally, a transcription termination and polyadenylation region.

16. The method of claim 15, wherein said subunit of the glycine decarboxylase complex is the H-protein (glycine cleavage complex lipoylprotein).

17. The method of claim 16, wherein said H-protein is an H-protein derived from a plant such as a seedbearing plant including Aegilops tauschii, Arabidopsis lyrata, Arabidopsis thaliana, Beta vulgaris, Brachypodium distachyon, Cicer arietinum, Cucumis sativus, Flaveria anomala, Flaveria bidentis, Flaveria brownii, Flaveria chlorifolia, Flaveria cronquistii, Flaveria floridana, Flaveria linearis, Flaveria palmeri, Flaveria pringlei, Flaveria pubescens, Flaveria trinervia, Glycine max, Hordeum vulgare subsp. vulgare, Lotus japonica, Medicago truncatula, Oryza sativa Indica Group, Oryza sativa Japonica Group, Pinus pinaster, Pisum abyssinicum, Pisum fulvum, Pisum sativum subsp. elatius, Pisum sativum subsp. transcaucasicum, Pisum sativum var. pumilio, Pisum sativum var. tibetanicum, Pisum sativum, Populus tremuloides, Populus trichocarpa, Ricinus communis, Sonneratia alba, Sorghum bicolor, Sorghum bicolor, Triticum aestivum, Triticum urartu, Vitis vinifera or Zea mays.

18. The method of claim 16, wherein said H-protein is an H-protein derived from an algal species including Micromonas or Chlamydomonas.

19. The method of claim 16, wherein said H-protein comprises an amino acid sequence having at least 80% sequence identity with the amino acid sequence of SEQ ID NO. 1.

20. The method of claim 16, wherein said subunit of the glycine decarboxylase complex is selected from the P-protein, the T-protein or the L-protein.

21. The method of claim 15, wherein said plant expressible promoter is a light-inducible promoter.

22. The method of claim 21, wherein said light-inducible promoter is selected from a promoter of a LS1 gene, a promoter of Rubisco small subunit gene, or a promoter of a chlorophyll a/b binding protein gene.

23. The method of claim 21, wherein said light-inducible promoter comprises the nucleotide sequence of SEQ ID NO. 3 from nucleotide 1 to nucleotide 1571.

24. The method of claim 15, wherein said recombinant gene comprises a ST-LS1 promoter from Solanum tuberosum operably linked to a H-protein encoding region from Flaveria pringlei.

25. The method according to claim 16, wherein said plant is selected from oilseed rape, cotton, rice, soybean, wheat, sugarcane or corn.

26. A method for increasing yield and/or biomass of a plant comprising the step of providing the cells of said plant with a recombinant gene according to claim 14.

27. The method of claim 26, wherein said plant is selected from oilseed rape, cotton, rice, soybean, wheat, sugarcane or corn.

28. A method for producing a plant with increased biomass or yield comprising the step of providing the cells of said plant with a recombinant gene according to claim 14 and optionally regenerating cells of said plant into a plant.

29. (canceled)

30. (canceled)

31. A seed of the plant of claim 1, comprising a recombinant gene according to claim 14.

32. A method for producing food, feed or biofuel or an industrial product comprising the steps of
a. obtaining a plant according to any one of claims 1 to 13 or a seed of claim 31; and
b. preparing the food, feed or industrial product from the plant or part thereof.

33. (canceled)

34. (canceled)


SEQUENCE LISTING (7 sequences)

SEQ ID NO: 1 - protein, 162 aa, Flaveria pringlei
MALRIWASSTANALRLSSATRPHFSPLSRCFSSVLDGLKYANSHEWVKHEGSVATIGITDHAQDHLGEVVFVDLPEAGGSVTKATGFGAVESVKATSDVNSPISGEIVEVNSKLSETPGLINSSPYEDGWMIKVKPSNPSELDSLMGAKEYTKFCEEEDSAH

SEQ ID NO: 2 - DNA, 694 bp, Flaveria pringlei
  misc_feature (60)..(548): coding region
ctttttcaacaccctaacacacacacaatcacacacacacacacacacttgcagactaaatggctcttagaatctgggcttcttccacagccaatgctctaaggctatcctctgctactagacctcatttctcacctctttccagatgcttctcttcagttttggatggattgaagtatgcaaattcacatgagtgggtgaagcatgaaggttctgttgctactattggtatcactgaccatgctcaggatcatcttggggaggtggtgtttgtggatttaccagaagccggtggctctgtgaccaaagccaccggctttggtgcagtagaaagtgtcaaagccaccagtgacgttaactccccgatctctggagagatcgttgaggtgaactcgaagttgtctgagactcctggtttgatcaattcgagcccgtatgaagatggatggatgattaaggtgaagccaagcaacccgtcggagctggattctttaatgggtgcaaaagagtacacaaagttctgcgaagaagaagattctgctcactagacagacatgaacataaatcatgtctcatgatttttttttttttcgtttcatcctcgacttcgactaactgaaatctcagtcgttcaataatcatattgtttcaaggtttgatcaatttatctttaaattatgctacaagttggaac

SEQ ID NO: 3 - DNA, 3699 bp, Solanum tuberosum
  misc_feature (20)..(27): sequence homologous to enhancer core sequence
ccaagtttttttgttgtttctatccactgatgtctgtgctactgaacataatgcatattgggggggggggggttgtgttcatctgttggacaagctaataatatctatttgttgtctgaatcacaattttcccatgataaattaaagccaaaattcggcttctcacaacctgatagaatctaattttatgacttttatcaaaaaaaagaagaagatagaatctaattttatgacatttaatggtacccattccttaaaaattcccgattcaactaatccttggagttcattttctcataggtggatttcatgctggagttactgtaaatagggttggatttctacattcttgcgagagtgatctgcattccagccccctctgaagcacaggagtcagctgcatttcaaatgattacccaattacagactatggcagaccgcacccattagatttaatgtggcaaagaaacatcaactccaatatttttgaatcctattaatggctatcataccatagtactgtattagtactgtccagaagcaaaaattgttgttttagaatattacttggttgcaaccgtatcagtgctttgaacatttatcaaggccaaatacataatggtgccttgaaatcaactctagtttattgaaatgatgtctattgagcacctatatgtgttgccagtattgcccatgcttaacttagcattctgttaacaagtgttatgctggatgttcgaatcgaacatggagttttcatgttgcttagaactatatgctttcagtgttagcagtatcggaaggtaaatacagtttaaacaccaaaagtagagagaaataaggaggaatttaatctttggatttgattcgtctttcttgtgggaatggtgaactcgttaaccagctaggtgaaatgaactcaatacttagttcaaaattgtttaagttccatttttggttaagagaattgagttgtgctctctagcttttgaagatgatttaggttcaaggactataattttacacgatattagagctagatccattgataggccgcgtttcttaatgtttttggccttgtaattctgttttcacattgattgactgaatgagttgtgctctttctatatgatctcggataattctttttctcatgagctaacttaagttaggatatgagctaactggtctccaacaggcaacaacccatagaggaaccaatcagttttttctttttgtcctcctcttccttgcacgagtggacttagtaaatagaaaggaaaatggcaaaaatgtataagaaacaagtcaatctcttgggtcccttaataatcaaagaaaaccaatgaatgaactccaagtgcacaaaactaagataaggcaaaggaaaataatttgtagtaagaatgctatatattgataatgttggtaataatattatctagaagcaatccacagaaggggtgccacgtgtcaacttgtaaatcacaaattgcaagtcatcactaatattattatcaagggaccatgcaaagtgaaaataaataattcatactaagtagtgagagcaaaagaagaaaaaaggtataggagaaaatggcaagcacagtaatgagctcattgagcctaaagccaactttcactttggagaaaacatcagtgaaagggcttccatcacttgctagatcttcttcttccttcaaagttgtggctagtggtgtcaagaagcttaagactgacaaaccctatggtatgtaattttctattcttgccacttatttaattgaattagagtttaacttacatacccaaacaatattacaatatttacattattagtgtagttttatttgtcttgataggtaatttatcatgtccatgtttactattagaattccttgtgatttagcttttaagtcgtgaccttataaatagtatatgaaatattttatgttgtcgatgtatatattcgtcaaactcttggaaaaaggtgcatatttactcatgtagtaatgacatgacttaattgtatcatctggtgtattttggggctatatttatacgcttgttgttagcaaaccgtctcgtatttgtccttgatgttaatttgagctctagtttagattgagaggatttcaggtgtcagaatacttaagtaaaaaagttcaaatttaccttagcaactttgactatggtttaaccaattaattacctgctagtctaatatacttcagattggagctatgttagggtcaagggctaatataagacaatatgttaacacaaggatatgaatatctgctaagtatagcggaagataaaattaagataattggtatagagagagagagagagagagagtagaggtgcttttaatccttgttctctcaactctttgatgaaagcagctcggtataccaagcatcccatattagtaggatacgagaaagggccacagcctaagagatgtgatgtagacaatctatcctaatgcaaattatgtctatatatttgaaaaaaccagagataaaatattgtaatgcaaaattaatactacctgctgattatgaagtgggctttgttactaggaattaatggaagcatggccttgagagatggcgttgatgcctcaggcaggaagcccaaggtttgtttctgcttctacctttgatatatatataataattatcattaattagtagtaatataatatttcaaatatttttttcaaaataaaagaatgtagtatatagcaattgcttttctgtagtttataagtgtgtatattttaatttataacttttctaatatatgaccaaaacatggtgatgtttagggaaagggtgtgtaccaatatgttgacaaatatggagctaatgttgatggatacaggtttgttaaaaacttcttaatttgtttaatgtaatgcttcactaatagaaaaatgaaacaaataatctaatactttgtgtgattttgaaaatgcagtcccatctacaacacagatgagtggtctccaagtggtgatgtctatgttggaggtatgacaatttactcgaacttccttttttaactcgaactatgtatatacacaacaacgttaataattaagtcgtactcattttgaatctactgactctagatcctgattcacacatgtaatataattgcaggtactactggcttagccatatgggccgtgaccttggttggtattcttgcaggaggtgctctccttgtctacaacacaagtgctttggcacagtagatgttatcctgtgttgtactattttaagttgtattcgagctcgcatcatgtaatcttttcaaaaatacttcaaaatatttcttggcatcttgattaatatgtaattgctgtgcttttactgtggataatattacacctaatgttgctgttttaggttcttataatcttaatctttaagtttagtcgtgattgattgccttccatgagactccacacagttataggctaatccaagatttgaagtgtaaggttcctgcaacgatctcaaattaattacaacaataatatatgagttcacagacaaatatttagtagattcttaatgcatagagctaatgggataataataccaacgcaacactagatggtacttccaatgcaaatgttacaagcaaaaatcttcatttgcctctggcaaagaatcatcaaaa

SEQ ID NO: 4 - DNA, 27 bp, Artificial Sequence
  oligonucleotide primer FpGLDH-SacI-S
gagctcatggctcttagaatctgggct

SEQ ID NO: 5 - DNA, 28 bp, Artificial Sequence
  oligonucleotide primer FpGLDH-EcoRI-AS
gaattcctacgtgagcagaatcttcttc

SEQ ID NO: 6 - DNA, 27 bp, Artificial Sequence
  oligonucleotide primer ST-LS1-SacI-S
gagctcggcttgatttgttagaaaatt

SEQ ID NO: 7 - DNA, 26 bp, Artificial Sequence
  oligonucleotide primer ST-LS1-BamHI-AS
ggatcctttctcctataccttttttc